(19) United States
(12) Patent Application Publication
(10) Pub. No.: US 2021/0072143 A1
(43) Pub. Date: Mar. 11, 2021

(54) HIGH CAPACITY MOLECULE DETECTION

(71) Applicant: Davinci Bio, Inc., San Francisco, CA (US)

(72) Inventors: Wei Zhang, Fremont, CA (US); Tao Hong, Santa Clara, CA (US); Zhisong Tong, Lafayette, CA (US)

(21) Appl. No.: 16/918,958

(22) Filed: Jul. 1, 2020

Related U.S. Application Data
(60) Provisional application No. 62/925,197, filed on Oct. 23, 2019.
(60) Provisional application No. 62/869,502, filed on Jul. 1, 2019.

(51) Int. Cl. G01N 15/14

(52) U.S. Cl. CPC G01N 15/1475; G01N 2015/1497; G01N 2015/1488; G01N 2015/1472

(57) ABSTRACT

The present disclosure relates generally to compositions and methods for high capacity detection of biological samples. Multiple optical labels as well as their combinations, which may include different ratios of the optical labels, can be used to allow detection of a large number of target molecules, cells, or tissues.

### CROSS REFERENCE TO RELATED APPLICATIONS

This application claims the benefit under 35 U.S.C. § 119(e) of U.S. Provisional Application Ser. Nos. 62/869,502, filed Jul. 1, 2019, and 62/925,197, filed Oct. 23, 2019, the content of each of which is incorporated by reference in its entirety into the present disclosure.

### STATEMENT REGARDING FEDERALLY SPONSORED RESEARCH OR DEVELOPMENT

This invention was made with government support under numbers 1R43GM130223-01 and 1R43GM136063-01 awarded by The Small Business Innovation Research (SBIR) of the National Institute of Health (NIH). The government has certain rights in the invention.

### SEQUENCE LISTING

The instant application contains a Sequence Listing which has been submitted in ASCII format via EFS-Web and is hereby incorporated by reference in its entirety. Said ASCII copy, created on Oct. 16, 2020, is named 298466_ST.txt and is 3,274 bytes in size.

### BACKGROUND

Fluorescent in-situ hybridization (FISH) is a powerful tool to detect individual DNA locus in-situ. It has been used widely in clinical diagnostics such as cytogenetics analysis. It is still considered the gold standard for detecting genomic abnormalities in clinic due to its high sensitivity (>98%), high specificity (>98%), broad coverage of detecting a wide range of both numerical and structural changes, and superior capability of detecting changes at single cell level. This is true even when next generation sequencing becomes routine for genomics analysis. Although FISH offers unique benefits for cytogenetic analysis because it can analyze genomic changes at the single cell level, the current FISH technology requires long hybridization time (from a few hours to overnight), lacks multiplex capability, and has low genomic resolution (˜100 kb). In particular, its limited multiplex capability is due to the limited number of different fluorescent colors/channels available for microscopic examination. Also, its low genomic resolution may arise from poor probe design. With such limitations, FISH has not been successfully used in single cell structural variation detection at the whole genome level.

For DNA analysis, multiple methods have been developed for multiplex DNA FISH such as Multiplex-FISH (M-FISH), COBRA-FISH, spectral karyotyping (SKY). These methods are mainly used for 24 color human chromosome visualization. Both M-FISH and SKY use a combinatorial labeling strategy. COBRA-FISH brings together combinatorial labeling with ratio labeling. Ratio labeling developed for chromosome analysis relies on a large amount of double-stranded DNA probes and fluorophores (typically 10,000-100,000) for each DNA locus and chromosome, and achieves a very low genomic resolution (10Mb-100Mb).

For RNA analysis, scRNA-seq is widely used for single-cell gene expression profiling at the whole transcriptome level. However, this method has significant limitations, such as the inability to provide spatial information, low detection efficiency and low cell capture efficiency. In-situ RNA detection is the best way to integrate sequence information and spatial information seamlessly without cell loss. It preserves tissue integrity and can be used to (1) visualize localized gene expression in single cells from both solid tissues and fluid specimens, (2) study gene regulation within the context of normal tissue architecture, (3) understand cellular heterogeneity at the transcriptome level, and (4) detect rare cell populations and pathogen infections.

A number of multiplexed and in-situ RNA detection technologies have been developed. They can be divided into three main categories, (1) in-situ sequencing methods, such as FISSEQ, (2) a hybrid approach with in-situ hybridization and in-situ sequencing, such as STARmap, (3) sequential FISH methods, such as seqFISH and MERFISH.

Although in-situ sequencing has the highest genomic resolution (single nucleotide), it takes days to weeks to perform a single experiment. Moreover, it can detect only ˜500 genes per cell due to its low detection efficiency (0.01%-5%) and the large dot size of rolling-cycle amplification, making it impossible to quantify gene expression accurately in single cells. STARmap integrates hydrogel-tissue chemistry, targeted signal amplification and in-situ sequencing to achieve multiplex RNA in-situ detection, but its detection efficiency is still not as high as the gold standard, single molecule RNA FISH, which uses multiple short DNA oligonucleotide probes to target one RNA copy at a time to achieve high detection specificity and sensitivity (>90%).

Multiplex and sequential FISH technologies improved the detection efficiency, of which two have the best performance, seqFISH and MERFISH. These two methods are both based on single-molecule RNA FISH to achieve >90% detection efficiency. However, their turnaround time is not fast enough, especially for a small gene panel (<500 genes) detection, which requires 3-5 days to complete. Their multiplex capability per round is still limited by the available fluorescence colors/channels.

For multiplex protein detection in-situ, several methods have also been developed as well such as Codex and the DNA exchange method. However, these methods are time consuming for a large protein panel detection. One of the main speed limiting factors is also the limited number of fluorescent channels available for a microscope.

For high capacity and high-throughput cell barcoding, multicolor and multi-intensity levels have been combined to detect different cell populations. Fluorescent cell barcoding (FCB) has been developed to differential up to 36 cell populations with two fluorophores by flow cytometry and has a capability to detect up to 216 cell populations with three fluorophores. However, the probe labeling scheme for FCB can't be expanded to in-situ molecule detection and spatial imaging.

### SUMMARY

The present disclosure, in various embodiments, provides compositions and methods for high capacity detection of multiple target molecules, particles and cells, which are spatially separated, under in-situ, in vitro, ex vivo or in vivo conditions. Molecules can include DNA, RNA, peptides, proteins, and etc, without limitation. To overcome the limitation of number of dyes of different colors, the present technology employs combinations of dyes, or more generally optical labels, at different ratios and intensities which are large in number and still are discernable from one another optically. The present technology further provides methods for design of label coding schemes and signal detection and correction.

In accordance with one embodiment of the present disclosure, provided is a sample prepared for examination, comprising: a first plurality of probes bound, directly or indirectly, to a first target molecule in a biological sample, and a second plurality of probes bound, directly or indirectly, to a second target molecule in the biological sample, wherein each target is associated with at least two kinds of optical labels, such that (a) the first plurality of probes is attached with at least a first kind of optical labels, the second plurality of probes is attached with at least a second kind of optical labels, and (b) the first and second target molecules, upon excitation, are associated with different ratios of signal intensities from two or more than two color channels.

Also provided, in one embodiment, is a kit, package, or mixture of probes for hybridization, comprising: a first plurality of probes each of which can bind to a first target molecule, or a first plurality of probes and one or more intermediate probes which allow the first plurality of probes to bind indirectly to the first target molecule, and a second plurality of probes each of which can bind to a second target molecule, or a second plurality of probes and one or more intermediate probes which allow the second plurality of probes to bind indirectly to the second target molecule, wherein each target is associated with at least two kinds of optical labels, such that (a) the first plurality of probes is attached with at least a first kind of optical labels, the second plurality of probes is attached with at least a second kind of optical labels, and (b) the first and second target molecules, upon excitation, are associated with different ratios of signal intensities from two or more than two color channels.

In yet another embodiment, provided is a method of detecting two or more target molecules in a sample, comprising admixing the probes to a sample that comprises the first and second targets under conditions to allow the probes to bind to the targets, wherein the different colors or color intensities associated with the targets allow detection of the targets.

Another embodiment provides a sample prepared for examination, comprising: a first plurality of probes bound, directly or indirectly, to a first target molecule in a biological sample, and a second plurality of probes bound, directly or indirectly, to a second target molecule in the biological sample, wherein each of the probes is attached with one or more optical labels such that: (a) at least a first optical label is associated with both the first and second target, but the first and second targets are associated with different numbers of the first optical label, and (b) the first and the second targets, upon excitation, are associated with different colors emitted from the optical labels, different intensities of a color, or the combination thereof.

Also provided, in one embodiment, is a kit, package, or mixture of probes for hybridization, comprising: a first plurality of probes each of which can bind to a first target molecule, or a first plurality of probes and one or more intermediate probes which allow the first plurality of probes to bind indirectly to the first target molecule, and a second plurality of probes each of which can bind to a second target molecule, or a second plurality of probes and one or more intermediate probes which allow the second plurality of probes to bind indirectly to the second target molecule, wherein each of the probes is attached with one or more optical labels such that, upon binding to the targeted biomolecule: (a) at least a first optical label will be associated with both the first and second target, but the first and second target will be associated with different numbers of the first optical label, and (b) the first and the second targets, upon excitation, are associated with different colors emitted from the optical labels, different intensities of a color, or the combination thereof.

Another embodiment provides an optical probe detected sample, comprising: a first plurality of probes bound, directly or indirectly, to a first targeted biomolecule, and a second plurality of probes bound, directly or indirectly, to a second targeted biomolecule, wherein each of the probes is attached with one or more optical labels such that: (a) the first and second targets are associated with two different optical labels, and (b) the first and the second targets are associated with two different intensities of the same color.

Yet in another embodiment, the present disclosure provides a probe set comprising a first probe and a second probe, wherein the first probe and the second probe are respectively attached with two optical labels having the same or overlapping color spectra but different intensities, wherein the first probe and the second probe each is further attached with a second optical label, and wherein the first probe and the second probe are distinguishable optically. In some embodiments, the probes have different binding specificities. In some embodiments, the first probe and the second probe are bound, directly or indirectly, to two target molecules in a biological sample.

Methods of detecting two different probes in a sample are also provided, wherein the first probe and the second probe are respectively attached with two optical labels having the same or overlapping color spectra but different intensities, the first probe and the second probe each is further attached with a second optical label, the methods comprising optically detecting and distinguishing the first and the second probes through two common color channels.

Yet in another embodiment, the present disclosure provides a probe set comprising a first probe and a second probe, wherein the first probe and the second probe are respectively attached with a first and second optical label having the same or overlapping color spectra but different intensities in two or more than two common color channels, and wherein the first probe and the second probe are distinguishable optically. In some embodiments, the probes have different binding specificities. In some embodiments, the first probe and the second probe are bound, directly or indirectly, to two target molecules in a biological sample.

Methods of detecting two different probes in a sample are also provided, wherein the first probe and the second probe are respectively attached with two optical labels having the same or overlapping color spectra but different intensities in two or more than two common color channels, the methods comprising optically detecting and distinguishing the first and the second probes through two or more than two common color channels.

In another embodiment, provided is a biological sample prepared for examination, comprising: a first target molecule bound, directly or indirectly, to a first optical label, and a second target molecule bound, directly or indirectly, to a second optical label, wherein the first target molecule and the second target molecule are optically distinguishable in one or more color channels by virtue of (a) different numbers of optical labels bound to each if the first optical label is the same as the second optical label, or (b) different intensities of similar color between the first optical label and the second optical label.

Also provided, in one embodiment, is a biological sample prepared for examination, comprising a plurality of distinct target molecules, each of which is bound to one or more optical labels, wherein the target molecules is optically distinguishable from one another in one or more color channels, and at least two of the target molecules are bound to the same optical labels but are optically distinguishable due to different ratios of the different optical labels bound to the target molecule.

Also provided, in one embodiment, is a biological sample prepared for examination, comprising a plurality of distinct target molecules, each of which is bound to one or more optical labels, wherein the target molecules is optically distinguishable from one another in one or more color channels, and at least two of the target molecules are bound to one common first optical label and, respectively, a second optical label and a third optical label having similar color, but are optically distinguishable due to the second and third optical labels having different intensities.

Also provided are kits, packages, or mixtures of probes for hybridization, comprising probes labeled with optical labels suitable for preparing a sample of the present disclosure. Still further provided is a method of detecting two or more target molecules in a sample, comprising admixing the probes of the disclosure to a sample that comprises target molecules under conditions to allow the probes to bind to the target molecules, wherein different kinds of target molecules are associated with different combinations of optical labels, each target molecule is detected by at least two color channels.

### BRIEF DESCRIPTION OF THE DRAWINGS

FIG. 1 present a schematic of HC-smFISH of an 8 intensity code matrix by 2 colors and 2 intensity levels. (a) Schematic of a probe labeling scheme for intensity ratio coding, either by a set of single dye labeled probes or probes with multiple dyes per probe. (b) The color channel scheme for intensity coding. Different dyes are imaged with different color channels (e.g. Cy3 and Cy5 dyes). Alternatively, dyes with overlapped emission spectra (e.g. Cy5, Cy5.5) are imaged with the same color channel. At least two color channels are used for intensity coding. Each rectangular bar represents one color channel. (c) The intensity coordinate map for an 8-code matrix (N=2 and M=2). 2 intensity levels (excluding 0) are used in each color channel. Each dot represents a measured intensity ratio from an oligo FISH spot. (d) A reference intensity code map generated from 2 dimension (2D) intensity distribution of FISH spots. Three clusters with boundaries are generated by analyzing the density distribution of spots to represent three intensity codes here. Each intensity code has an irregular boundary.

FIG. 2 illustrates Labeling Scheme-1. (a) Direct labeling. (b) Indirect labeling. (c) Indirect labeling by branched DNA amplification. Two color channels are used here: Cy3/600 and Cy5/700 channel. The first digit of intensity codes represents the intensity level in Cy3 channel. The second digit of intensity codes represents the intensity level in Cy5 channel.

FIG. 3 illustrates Labeling Scheme-2. (a) Direct labeling. (b) Indirect labeling. (c) Indirect labeling by branched DNA amplification. Two color channels are used here: Cy3/600 and Cy5/700 channel. The first digit of intensity codes represents the intensity level in Cy3 channel. The second digit of intensity codes represents the intensity level in Cy5 channel.

FIG. 4 illustrates Labeling Scheme-3. (a) Direct labeling. (b) Indirect labeling. (c) Indirect labeling by branched DNA amplification. The first digit of intensity codes represents the intensity level in Cy3/600 channel. The second digit of intensity codes represents the intensity level in Cy5/700 channel.

FIG. 5 illustrates an alternative signal amplification approach by rolling cycle amplification to achieve various intensity codes for nucleic acid based target detection. (a) An example of different components of the probe labeling for rolling cycle amplification. (b) Imaging probes with various intensity codes. (c) The signal amplification scheme using one target as an example. Each unique target is labeled by a target-unique primer, target-unique padlock probe with a target-unique identifier. The target-unique identifier is amplified by rolling cycle amplification. Finally, imaging probes with a target-unique combination of dyes are hybridized with the amplified identifiers to achieve target-specific intensity coding. By attaching imaging probes with different number and kinds of dyes, a variety of intensity codes can be obtained.

FIG. 6 illustrates adding a reference dye for error-correction of non-specific binding. Cy3, Alexa 546, Cy5, Cy5.5, are used for intensity coding. Alexa 488 is used as a reference dye to remove non-specific binding signal. (a) Adding a reference dye for the Labeling Scheme-2. (b) Adding a reference dye for the Labeling Scheme-3. Three color channels are used here: Alexa488/500 channel, Cy3/600 and Cy5/700 channel. 500 channel is not used for intensity coding. The first digit of intensity codes represents the intensity level in Cy3 channel. The second digit of intensity codes represents the intensity level in Cy5 channel.

FIG. 7 illustrates differentiated labeling approach for Labeling Scheme-2 and Scheme-3. Different dyes are associated with different primary probes (probes binding to targets directly). (a) Differentiated labeling for the Labeling Scheme-2. (b) Differentiated labeling for the Labeling Scheme-3. The first digit of intensity codes represents the intensity level in Cy3 channel. The second digit of intensity codes represents the intensity level in Cy5 channel.

FIG. 8 illustrates an alternating labeling approach for differentiated labeling. Primary probes associated with different labels are designed to bind with the same target in an alternating way. The first digit of intensity codes represents the intensity level in Cy3 channel. The second digit of intensity codes represents the intensity level in Cy5 channel.

FIG. 9 illustrates the Alternative Labeling Scheme-3: one dye per target but different targets associated different dyes with overlapped spectra in at least two color channels. (a) One dye per target labeling scheme. Three intensity ratio codes are generated by three dyes with 2 color channels. (b) An illustration of 3 clusters of different intensity ratio codes generated by three dyes with overlapped spectra. Each cluster is cut off by an intensity threshold at the lower intensity end to remove background and non-specific binding signal. The first digit of intensity codes represents the intensity level in Channel A. The second digit of intensity codes represents the intensity level in Channel B.

FIG. 10 illustrates the overlapped emission spectra of Alexa 647 and Alexa 700. Two color channels can be used to generate two intensity ratios for Alexa 647 and Alexa 700 so that these two dyes can be distinguished by these two color channels. (a) Matching 2 dyes with 2 targets and a pair of two color channels. (b) The overlapped spectra of Alexa 647 and Alexa 700 in two color channels. Channel A: 650 nm to 710 nm. Channel B: 745 nm to 805 nm.

FIG. 11 illustrates the Labeling Scheme-4 by quenching. Intensity levels and intensity variations can be finely adjusted by various quenching designs, such as FRET (d-h), self quenching of accumulating more of the same dye in a limited distance (i, j, 1), and quencher (k and m).

FIG. 12 illustrates protein labeling by intensity coding. (a) A protein target attached with a target-specific oligo sequence as a primary probe. (b) Illustration of protein coding by Labeling Scheme-2. (c) Illustration of protein coding by Labeling Scheme-3. (d) Alternative signal amplification by rolling cycle amplification. In FIG. 12(d), a target such as a protein is labeled by a target-specific antibody attached with a target-unique oligonucleotide. Antibodies can be replaced by other target-specific linkers such as small epitopes like HA or SNAP tags. A target-unique padlock probe with a target-unique identifier is hybridized with the oligo on the antibody and then circulated by ligation. The target-unique identifier is amplified by rolling cycle amplification. Finally, imaging probes with a target-unique combination of dyes are hybridized with the amplified identifiers to achieve target-specific intensity coding. By attaching imaging probes with different number and kinds of dyes, a variety of intensity codes can be obtained. 3 intensity codes (1:0, 1:1, 3:1) are illustrated in the left of the figure.

FIG. 13 illustrates different applications of intensity coding. (a) Detecting different cell types. Different numbers represent different cell types with distinct cell markers. (b) Detecting proteins randomly distributed on a solid scaffold such as a glass slide. Different kinds of proteins are associated with different combination of optical labels. (c) Detecting multiple targets simultaneously with a spatial pattern on microarray. Different targets at different spatial locations are encoded with probes programmed with different intensity ratios.

FIG. 14 shows the results of single molecule RNA FISH with Oligo DNA Probes and the simulation of 2 color intensity coding. (a) Images are TFRC RNA FISH in HeLa cells. The image on the right is zoomed-in from the selected region of the image on the left. (b) Intensity distributions of TFRC FISH spots. Ratio 1:0 is the experimental result from 790 spots detected by single Cy3 labeled TFRC probes. Ratio 2:0 and 3:0 are simulated data based on Ratio 1:0 with double intensity and triple intensity per spot, respectively. Ratio 2:0 has 40% spots overlapping with Ratio 1:0. Ratio 3:0 has <5% spots overlapping with Ratio 1:0. (c-d) shows the results of a simulation of the intensity distribution for an 8 ratio coding scheme. (c) shows an 8 ratio coding scheme with N=2 and M=2 when the second intensity level has 2× (2 times) intensity of the first intensity level. (d) shows an 8 ratio coding scheme with N=2 and M=2 while the second intensity level has 3× intensity of the first intensity level.

FIG. 15 shows results of intensity ratio imaging with the Labeling Scheme-3. (a)-(b) Confocal imaging of telomere labeled with Cy5 (a) and Cy3 (b), a and b are rendered in the same display range of intensity. (c)-(d) Confocal imaging of centromere labeled with Cy5 (c) and Alexa532 (d), c and d are rendered in the same display range of intensity. (e)-(f) Intensity distributions of telomere and centromere probes with different dye pairs: (e) Tel-Cy5-Cy3, Cen-Cy5.5-Cy3, Cen-Cy5-A532 and (f) Tel-Cy5-Cy3, Cen-Cy5-Atto590, Cen-Cy5-A546. (e): Tel-Cy5-Cy3 overlaps with Cen-Cy5.5-Cy3 but is well separated from Cen-Cy5-A532. (f): Tel-Cy5-Cy3 overlaps with Cen-Cy5-Atto590 but is largely distinguishable from Cen-Cy5-A546. (g) Histogram of intensity ratios between the color channel of 700 and 600 for different dye pairs.

FIG. 16 shows results of RNA FISH by intensity coding using the Labeling Scheme-3. POLR2A is labeled with Cy5.5 and A546. CTNNB1 is labeled with Cy5 and Cy3. (a) An image of POLR2A and CTNNB1 co-labeling in 700 channel. (b) An image of POLR2A and CTNNB1 co-labeling in 600 channel. (c) An image of CTNNB1 labeling only in 700 channel. (d) An image of CTNNB1 labeling only in 600 channel. (e) An image of POLR2A labeling only in 700 channel. (f) An image of POLR2A labeling only in 600 channel. (g) Negative control without labeling of primary probes in 700 channel. (h) Negative control without labeling of primary probes in 600 channel. The amplifiers and imaging probes for both RNA targets are added in the negative control.

FIG. 17 shows the way of using the reference intensity code map to assign FISH spots with intensity codes. (a) A reference intensity code map generated by labeling CTNNB1 with Cy5 and Cy3, POLR2A with Cy5.5 and Alexa 546, separately. A boundary is plotted for each dye combinations. The cluster in the upper left represents the intensity distribution for POLR2A with the co-labeling of Cy5.5 and Alexa 546. The cluster in the lower right represents the intensity distribution of CTNNB1 with the co-labeling of Cy5 and Cy3. (b) 2D intensity map of Cell 1 with co-labeling of CTNNB1 and POLR2A. (c) 2D intensity map of Cell 2 with co-labeling of CTNNB1 and POLR2A. (d) 2D intensity map of Cell 3 with co-labeling of CTNNB1 and POLR2A. POLR2A is labeled with Cy5.5 and Alexa 546. CTNNB1 is labeled with Cy5 and Cy3.

FIG. 18 shows results of 4 RNA co-labeling by intensity coding. (a) An image of multiplexed labeling of MEF cells in 700 channel. (b) An image of multiplexed labeling of MEF cells in 600 channel. (c) The overlapped image of (a) and (b) with a zoomed-in area shown in (d). (d) A zoom-in image of the region selected in (c). Different shapes mark the positions of different RNA copies, where each copy of HOXB1 is marked with a circle, POLR2A with a square, TFRC with a diamond and CTNNB1 with a hexagram. (e) The 2D intensity distribution map in log scale where 4 clusters of dots are clearly distinguished from each other.

FIG. 19 shows results of intensity ratio based separation of 2 DNA loci in primary tissues (a-c: telomere and centromere loci in mouse brain tissue., d-f: chromosome-1 repetitive loci (Ch1-Re) and centromere loci in human PBMC cells). (a) Separation of two clusters of intensity ratios based on their resolvable intensity ratio distribution. (b)-(c) The identification results of telomere and centromere based on the assignment of intensity ratio codes (b) with a zoomed-in region in (c). (d) Separation of two clusters of intensity ratios based on their resolvable intensity ratio distribution. (e)-(f) The identification results of Ch1-Re and centromere based on the assignment of intensity ratio codes (e) with a zoomed-in region in (f).

FIG. 20 shows results of DNA FISH by different intensity coded probes. (a-b) Images in two color channels of telomere probes (1 Cy5 and 10 Cy3 per primary probe). (a): Cy5/700 channel, (b): Cy3/600 channel. (c) 2D intensity distribution of telomere labeling with one intensity ratio of 1 Cy5:10 Cy3. (d) 2D intensity distribution of telomere labeling with one intensity ratio of 1 Cy5:2 Cy3. (e) 2D intensity distribution of telomere labeling with one intensity ratio of 1 Cy5:30 Cy3. (c-e) shares the same reference line.

### DETAILED DESCRIPTION

The present disclosure, in various embodiments, describes compositions and methods for carrying out high capacity detection of molecules, molecular complexes, cells, or tissues, etc. The high capacity, in some embodiments, can be attributed to the use of a larger number of labeling machineries, as compared to what are routinely done, that can be distinguished from each other. Such labeling machineries, as demonstrated herein, can be single molecules or combinations of two or more molecules. Further, the difference between the labeling machineries may be a matter of different colors or spectra, different intensities, or both.

A. High Capacity Single Molecule FISH (HC-smFISH)

One embodiment of the present technology is illustrated in a “High Capacity Single Molecule FISH,” or HC-smFISH. HC-smFISH utilizes an intensity ratio coding scheme (or referred to as “intensity coding”, “color ratio coding”, or “spectral ratio coding”) at single molecule detection sensitivity and high genomic resolution (>20 nt). HC-smFISH enables visualization of significantly more RNA species or DNA loci than what can be achieved with the conventional FISH technology. In a conventional FISH technology, the number of RNA species or DNA loci that can be visualized simultaneously is limited by the number of different fluorescent colors available for the microscopic method used. With HC-smFISH, one can profile a large DNA/RNA panel and even the whole transcriptome or genome.

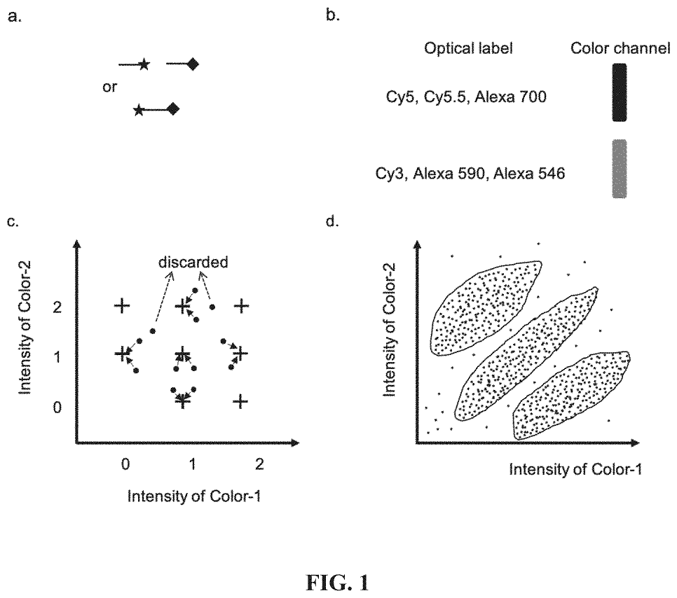

HC-smFISH uses the intensity ratio (or referred to as color ratio) from one or more color channels as intensity codes to differentiate RNA species or DNA loci. Due to intensity variation, typically, intensity ratios between two or more color channels are used for multiplex barcoding so that intensity overlap can be minimized across any two intensity codes. Theoretically, the number of intensity combinations or intensity ratio combinations available can be programmed by the oligo probe labeling schemes. For example, a labeling scheme of 2 colors and 2 intensity levels per color generates 8 intensity combinations (1:0, 2:0, 0:1, 0:2, 1:1, 1:2, 2:1, 2:2) and thus can encode up to 8 RNA species or DNA loci inside a cell. (FIG. 1).

Ideally, individual FISH spots from the same RNA species or DNA loci should have the same intensity combination or intensity ratio combination as they are detected by probes with the same labeling design. Experimentally, the intensity values generated by individual FISH spots from the same RNA species or DNA loci may deviate from the designated intensity code. By comparing the distance of each experimental intensity combinations from the designated combination, each FISH spot can be assigned to the nearest intensity code (FIG. 1c). For FISH spots with ambiguous intensity values (in the middle of two designated ratios), they will be discarded.

Theoretically, the capacity of HC-smFISH, that is, the maximum number of programmed ratios of a labeling scheme or the maximum number of RNA species or DNA loci detected per round of FISH, is determined by the number of fluorescent colors and intensity levels, which is predicted by formula: Pₘₐₓ=(M+1) ^{N}−1. Here, P represents the number of different targets such as RNA species or DNA loci that can be detected (i.e. the multiplex capability per round of FISH), M represents the number of intensity levels (excluding zero) per color as determined by the probe labeling scheme, N represents the number of fluorescent colors (i.e. color channels) available from a microscope. Thus, 2 fluorescent colors with 2 intensity levels (including the first intensity level (or called as intensity level 1) and the second intensity level (or called as intensity level 2), intensity level 0 is not included) per color can code up to 8 RNA species or DNA loci, and 4 colors with 3 intensity levels per color can code 255 RNA species or DNA loci. In the HC-smFISH design, one can use at least one non-zero intensity levels (M≥1) to produce a drastically larger code size per round of hybridization and imaging.

For intensity coding, the number of color channels defines the number of digits of each intensity code. If only one color channel is used for a code, such as 1:0, it is one digit code. If two color channels are used for a code, such as 1:2, the code is a two-digit code.

HC-smFISH, therefore, can achieve a 10-100× higher multiplex capability per round of FISH than other FISH technologies. Each round of HC-smFISH can image hundreds of RNA genes with 4-5 fluorescent colors.

For practical applications, such as profiling a large RNA panels for single cell gene expression analysis, HC-smFISH can combine the intensity coding scheme with an error-correction code. The basic principle is to design a set of codes that sparsely occupy the entire coding space so that miss-assignment in just one color channel can be detected and corrected. For example, for N=5 and M=3, Pₘₐₓ=1023 but choose P=200. Integration with coding across sequential rounds of hybridization, imaging and dehybridization allows further expansion of the detection capacity in a straightforward manner.

The number of combinatorial ratio codes can be predicted by this formula: Qₘₐₓ=((M+1)^{N})^{K}−1. Here, Q means the total number of combinatorial intensity ratio codes, M means intensity levels (excluding zero), N means the number of colors (i.e. color channels), K means the number of sequential rounds. Therefore, if M=3, N=5, Qₘₐₓ=1023, but if Q is chosen to be 200, 3 rounds of 5 color imaging can in principle produce combinatorial codes covering the entire human or mouse transcriptome (˜20,000 protein coding genes). Compared to MERFISH and seqFISH which have a much smaller value of P, the high multiplex capacity of HC-smFISH is particularly advantageous for the analysis of large-sized tissue because the long imaging time per round of analysis favors fewer rounds.

For best performance of using intensity coding, any two copy of nucleic acid targets should be separated spatially beyond the optical resolution of the imaging system when using intensity coding here. Typically, this optical resolution is around 250 nm when using visible optical labels on a conventional optical microscope. However, by advanced optical imaging methods, this optical resolution can be enhanced to less than 100 nm or even less than 20 nm so that more molecules can be detected in the same space and higher detection efficiency can be achieved with intensity coding.

B. High Capacity Detection for Other Kinds of Targets

HC-smFISH is an example implementation of the high capacity detection technology. Besides RNA and DNA, the high capacity detection technology can also be used to detect other non-nucleic acid based biomolecules, particles, cells and tissue samples that are spatially separated from each other, such as proteins, carbohydrates, lipids, complexes, cell organelles, cells, tissues, and microorganisms, etc. A general principle here is barcoding multiple targets with programmed oligonucleotides or peptides (each kind of target is programmed with a unique oligo or peptide sequence) first, then using intensity coded probe labeling schemes for HC-smFISH and hybridization based probes to detect the programmed oligos or peptides associated with various targets. Oligonucleotides can consist of natural nucleotides or unnatural nucleotides such as LNA. Peptides can consist of natural amino acids or unnatural amino acids. Alternatively, hybrid oligonucleotide and peptide sequences can be used.

For best performance of using intensity coding, any two copy of non-nucleic acid targets should be separated spatially beyond the optical resolution of the imaging system when using intensity coding here. Typically, the spatial resolution is more than 250 nm. In some cases, the resolution can be as low as 20 nm with a super-resolution imaging setup and algorithms.

C. Labeling Schemes for High Capacity Intensity Coding

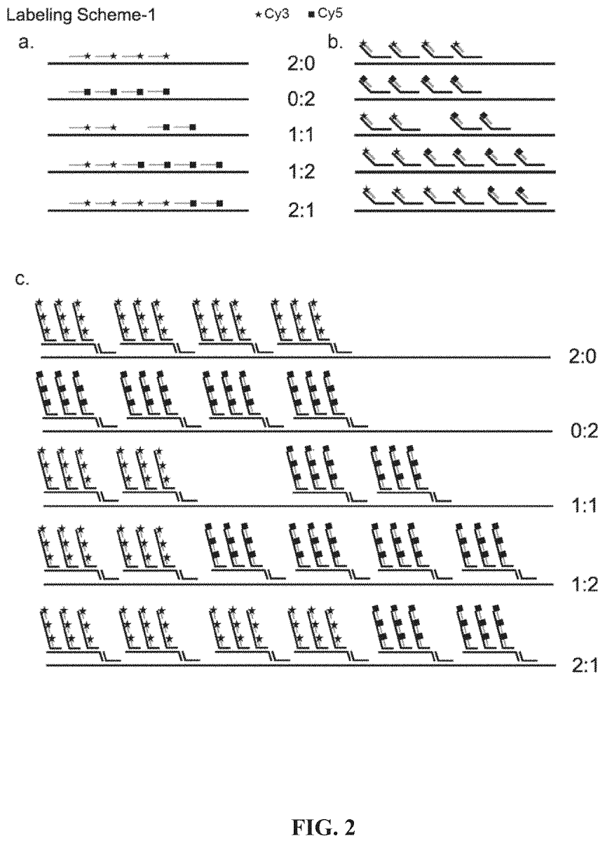

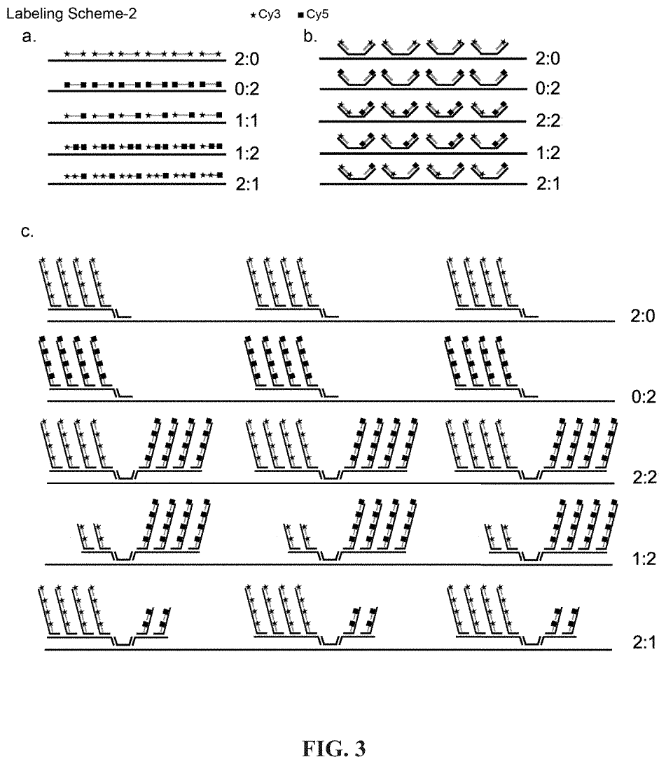

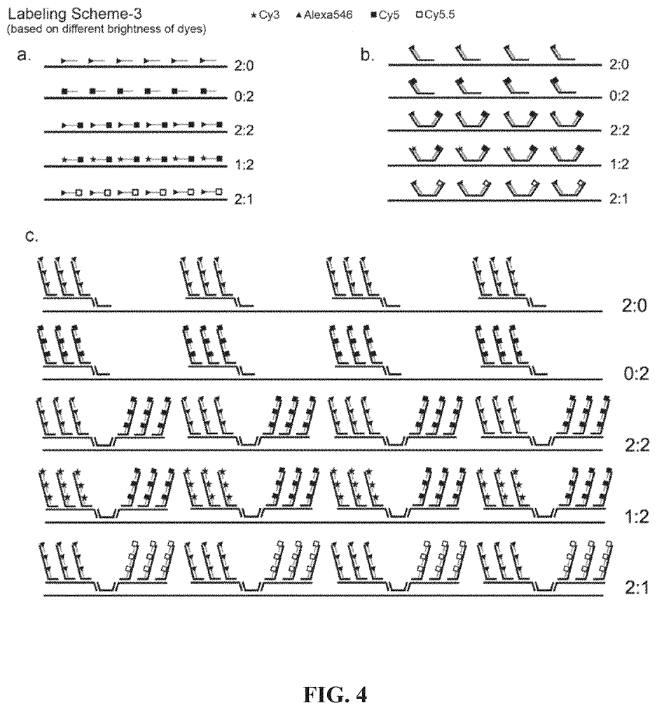

Three basic labeling schemes as illustrated in FIG. 2-4 are named as Labeling Scheme-1 (FIG. 2), Labeling Scheme-2 (FIG. 3) and Labeling Scheme-3 (FIG. 4), respectively. In each basic labeling scheme, three basic variations can be further implemented: direct labeling (the panel a of FIG. 2-4), indirect labeling without signal amplification (the panel b of FIG. 2-4), indirect labeling with signal amplification (the panel c of FIG. 2-4). In Labeling Scheme-1, the number of target-binding probes or primary probes, which are proportional to the number of optical labels for each optical label associated with a target, defines the intensity level in each color channel. In Labeling Scheme-2, the number of optical labels associated with a primary probe, instead of the number of primary probes, defines the intensity level. In Labeling Scheme-3, only the kinds of dyes associated with a target, instead of the number of optical labels or probes, defines the intensity level in each color channel.

As illustrated in FIG. 2-4, each horizontal long, bold and solid line represents a target for detection. The target may be a molecule (e.g., RNA, DNA, protein, carbohydrate, lipid), a complex (e.g., antigen-antibody complex, ligand-receptor complex, prothrombinase complex), an organelle, a cell, a tissue, or a microorganism.

Each target is bound, directly or indirectly, to a plurality of probes, which are represented by short solid lines. Multiple short lines may bind to a single long line, representing multiple probes bound to a target. Multiple short lines may bind to a single short line, representing multiple probes bound to an intermediate probe, as illustrated in the panel c of FIG. 2-4. Some solid lines are further coupled to one or more solid shapes (e.g., star and square) representing imaging probes attached with optical labels. Each shape represents a different optical label. For instance, as shown in FIG. 2, a dark star represents Cy3 which is a bright, greenish yellow fluorescent dye, and a dark square represents Cy5 which is a bright, far-red-fluorescent dye.

As illustrated in panels a of FIG. 2, each target is associated with a number of optical labels (e.g., Cy3 and/or Cy5). The targets of the first row (labeled “2:0”) can be easily distinguishable from the targets of the 2^{nd} row (labeled “0:2”). In panel a, the target in the first row is associated with 4 Cy3 dyes, and no Cy5 dye; the target in the second row is associated with no Cy3 dye but 4 Cy5 dye. Therefore, the first target would appear as bright greenish yellow in the Cy3 color channel under microscope and the second target would appear as bright red in the Cy5 color channel. The third target of the third row (labeled “1:1”) is associated with 2 Cy3 dye and 2 Cy5 dye. Therefore, the third target would show strong signals in both Cy3 and Cy5 channels but its intensity is significantly weaker than the first target and the second target in both channels. If we define the first intensity level is distinguished by 2 dyes in each color channel and the second intensity level 4 dyes in each channel, the intensity of each target above in different channels can be assigned with different intensity levels. Therefore, each target is encoded with a unique intensity code. For example, the third target has the first intensity level in both channels so that it is labeled by the intensity code 1:1. The first target has the second intensity level in Cy3 channel and no intensity in Cy5 channel, assigned with the intensity code 2:0. The second target has the second intensity level in Cy5 channel and no intensity in Cy3 channel, assigned with an intensity code of 0:2.

Still in panel a of FIG. 2, the targets in the 4^{th} row and the 5^{th} row are associated with both of the Cy3 and the Cy5 dyes. However, the fourth target in the 4^{th} row is associated with fewer Cy3 (2:4) and more Cy5 (4:2) than the fifth target in the 5^{th} row. The fourth target is assigned with an intensity code of 1:2. The fifth target is assigned with an intensity code of 2:1. Therefore, they can be distinguished, not purely by color, but by a combination of colors and their respective intensities.

In accordance with some embodiments of the present disclosure, therefore, provided is a sample prepared for examination, comprising a first plurality of probes bound, directly or indirectly, to a first target molecule, cell or tissue in a biological sample, and a second plurality of probes bound, directly or indirectly, to a second target molecule, cell or tissue in the biological sample. In some embodiments, each of the probes is attached with one or more optical labels such that at least a first optical label is associated with both the first and second target, but the first and second targets are associated with different numbers of the first optical label. Further, in some embodiments, the first and the second targets, upon excitation, are associated with different colors emitted from the optical labels, different intensities of a color, or the combination thereof, such that the first target and the second target can be distinguished optically.

The term “probe” as used herein, refers to a molecule or a group of aggregated molecules that are coupled to a detectable label, or is suitable for coupling to a detectable label indirectly. Non-limiting examples of probes include DNA or RNA oligonucleotides, unnatural oligonucleotides, peptide nucleic acids (PNA), antibodies, receptors, ligands, synthetic polymers such as dendrimers, and various chemical compounds. Typically, hybridization based nucleic acid probes, or hybridization based peptide probes such as PNA probes are used. The probe directly attached with an optical label is referred to as an “imaging probe”. An imaging probe may be associated with just one optical label. Alternatively, an imaging probe may be associated with two or more of the same kinds of optical labels. Alternatively, an imaging probe may be associated with two or more of different kinds of optical labels. For nucleic acid targets, the oligo or peptide probe directly bound with a target is referred to as a “primary probe”, or “target-binding probe”. For non-nucleic acid targets, the first probe associated with a target is referred to as a “primary probe” and then more optical probes (including imaging probes and intermediate probes) are associated with the first probe through a series of hybridization to detect the target.

The term “optical label” or simply “label” refers to a molecule or a biological moiety which, upon suitable excitation, can emit signals detectable by optical means. Examples include fluorescent dyes (such as Cy3, Cy5, Alexa 532), fluorescent proteins (such as GFP, YFP and RFP), chromogenic dyes, quantum dots and other kinds of nanoparticles. In some embodiments, the optical label is reactive and can be attached to another molecule, such as a probe. Examples include Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, G-Dye100, G-Dye200, G-Dye300, G-Dye400, Cy2, Cy3, Cy3B, Cy3.5, Cy5, Cy5.5, Cy7, TRITC, X-Rhodamine, Lissamine Rhodamine B, Texas Red, Allophycocyanin (APC), and APC-Cy7 conjugates. In some embodiments, the optical labels are photoswitchable dyes, such as photoswitchable rodamine amides. In some embodiments, optical labels are attached onto probes by chemical conjugation. In some embodiments, optical labels are attached onto probes by physical association only but not chemical reaction. In some embodiments, optical labels are attached onto probes by both physical association and also chemical reaction.

The term “color channel”, “color spectrum”, or simply “color”, “spectrum”, “channel” refers to a window of emission spectrum (e.g. from 650-700 nm), a window of excitation spectrum (e.g. 640 nm, or 635-645 nm), or a combination of these two optical properties. Alternatively, the term “color channel”, or “channel” refers to a combination of optical components to define or select the window of these two optical properties. E.g., these components can be, but not limited to, an emission filter (e.g. a filter with an emission bandpass window of 650-700 nm), an excitation filter (e.g. a filter with an excitation bandpass window of 635-645 nm), or a physical filter set including a dichroic mirror, an emission filter and an excitation filter. The number of color channels available, physically but not virtually achieved by pseudo-colors, are limited by the microscope system used. Typically, 3-5 color channels are available by a fluorescence microscope without crosstalk between adjacent channels based on the excitation and emission wavelengths and corresponding optical filters. Typically, in this patent application, we use 2-3 channels to image different optical labels: Cy5/700 channel to image Cy5, Cy5.5 dyes; Cy3/600 channel to image Cy3, Alexa 546, Alexa 532 dyes; Alexa 488/500 channel to image Alexa 488 dye. More specifically, in the Example section, Alexa 488/500 channel is equipped with an emission filter at 525/50 m, Cy3/600 channel with an emission filter at 600/50 m, Cy5/700 channel with an emission filter at 700/75 m.

In some embodiments, a biological sample for examination comprises a first plurality of probes bound, directly or indirectly, to a first target molecule, cell or tissue in a biological sample, and a second plurality of probes bound, directly or indirectly, to a second target molecule, cell or tissue in the biological sample. wherein a first optical label is associated with a first target, a second optical is associated with a second target, the number of the first optical label associated with the first target and the number of the first optical label associated with the second target differ by at least 2, 3, 4, 5, 6, 7, 8, 9, 10, 15, 20, 30 40, 50, 60, 70. 80, 90, 100, 200, 300, 400, or 500. Such differences can help ensure that the same colors (or similar colors) at different intensities (based on number of optical labels used) can be distinguished from one another. In some embodiments, the difference of numbers of optical labels to detect two different kinds of targets is not greater than 200, 500, 1000, 2000, 5000 or 10000.

In some embodiments, a biological sample for examination comprises a first plurality of probes bound, directly or indirectly, to a first target molecule, cell or tissue in a biological sample, and a second plurality of probes bound, directly or indirectly, to a second target molecule, cell or tissue in the biological sample. wherein a first optical label is associated with a first target, a second optical is associated with a second target, the ratio of the number of the first optical label associated with the first target and the number of the first optical label associated with the second target is at least 2, In some embodiments, the ratio of the number of the first optical label associated with the first target and the number of the first optical label associated with the second target is at least 2.5, 3, 4, 5, 5.6, 7, 8, or 10,

In some embodiments, the probes are single-stranded oligonucleotides or peptides. In some embodiments, the optical labels are fluorescent dyes, chromogenic dyes, fluorescent proteins, quantum dots or other kinds of nanoparticles. In some embodiments, the first and second target are further associated with a second optical label attached to the probes, but are associated with different numbers of the second optical label.

In some embodiments, the probes are chimeric polymers. In some embodiments, the chimeric polymer consists of natural polymer (such as nucleic acid or protein) and synthetic polymer. In some embodiments, the probes are synthetic polymers. In some embodiments, the probes are single strand oligonucleotides. In some embodiments, the probes are peptides. In some embodiments, the probes are peptide nucleic acids (PNAs). In some embodiments, the probes are oligonucleotides conjugated with peptides. In some embodiments, the probes are oligonucleotides attached with other synthetic polymers such as dendrimers. In some embodiments, the probes are peptides attached with other synthetic polymers such as dendrimers. In some embodiments, a probe may be part of a multi-strand complex.

In some embodiments, the single-stranded oligonucleotide probes may be comprised of DNA, RNA, or nucleic acid-like structures with other phosphate-sugar backbones (e.g., LNA, XNA). In some embodiments, the single-stranded probes consist of backbones comprising non-phosphate-sugar moieties (e.g. peptide nucleic acid and morpholino). In some embodiments, the single-stranded oligo probes may comprise secondary structure such as a hairpin loop.

In some embodiments, a plurality of probes associated with a target is comprised of only one probe. In some embodiments, a plurality of probes associated with a target is comprised of at least two probes of different sequences. In some embodiments, a plurality of probes associated with a target is comprised of at least 4, or 12 probes of different sequences.

In some embodiments, a plurality of primary probes associated with a target consist of at least one probe. In some embodiments, a plurality of primary probes associated with a target consist of at least two probes of different target-binding sequences. In some embodiments, a plurality of primary probes associated with a target consist of at least 5, 10, 20, 30, or 50 probes of different sequences. In some embodiments, a plurality of primary probes associated with a target consist of no more than 100 probes of different sequences.

In some embodiments, the first and second target are further associated with a second optical label attached to the probes, but are associated with the same number of the second optical label. In some embodiments, the first and second target are further associated with a second optical label attached to the probes, but are associated with different number of the second optical label. In some embodiments, the first target is associated with a third optical label attached to the probes, and the second target is not associated with the third optical label.

In some embodiments, each of the first plurality of probes is attached with a single optical label. In some embodiments, at least two of the first plurality of the probes are attached with two different optical labels. In some embodiments, at least two of the first plurality of the probes are attached with more than two different optical labels.

In some embodiments, each probe of the first plurality of probes is attached with two or more different optical labels or two or more different numbers of the same optical labels. In some embodiments, each probe of the first plurality of probes is associated with the same color.

In some embodiments, a plurality of probes associated with a target consist of primary probes only. In some embodiments, a plurality of probes associated with a target consist of two tiers of probes: primary probes and imaging probes (or named as secondary probes). In some embodiments, a plurality of probes associated with a target consist of more than two tiers of probes. In some embodiments, a plurality of probes associated with a target consist of 4 tiers of probes: primary probes, primary amplifiers (or named as primary amplification probes), secondary amplifiers (or named as secondary amplification probes) and imaging probes.

In some embodiments, a biological sample for examination comprises a first plurality of probes bound, directly or indirectly, to a first target molecule, cell or tissue in a biological sample, and a second plurality of probes bound, directly or indirectly, to a second target molecule, cell or tissue in the biological sample, wherein each target is associated with at least two optical labels, a first optical label is associated with a first target, a second optical is associated with a second target, the first and second optical labels have overlapped spectra in a first color channel, a third optical label is associated with both the first and second targets and detected by a second color channel; wherein the first and second target, upon excitation, are associated with different ratios of signal intensities from two color channels. In some embodiments, a fourth optical label is further associated with both the first and second targets, and the first and second target, upon excitation, are associated with different ratios of signal intensities from three color channels.

In some embodiments, a biological sample for examination comprises a first plurality of probes bound, directly or indirectly, to a first target molecule, cell or tissue in a biological sample, and a second plurality of probes bound, directly or indirectly, to a second target molecule, cell or tissue in the biological sample, wherein each target is associated with at least two optical labels, a first and a second optical label are associated with a first target, a third and a fourth optical label are associated with a second target, the first and third optical labels have overlapped spectra in a first color channel, the second and fourth optical labels have overlapped spectra in a second color channel; wherein the first and second target, upon excitation, are associated with different ratios of signal intensities from two color channels. In some embodiments, a fourth optical label is further associated with both the first and second targets, and the first and second target, upon excitation, are associated with different ratios of signal intensities from three color channels. In some embodiments, a fifth optical label is further associated with both the first and second targets, and the first and second target, upon excitation, are associated with different ratios of signal intensities from three color channels.

In some embodiments, a biological sample for examination comprises a first plurality of probes bound, directly or indirectly, to a first target molecule, cell or tissue in a biological sample, and a second plurality of probes bound, directly or indirectly, to a second target molecule, cell or tissue in the biological sample, wherein each target is associated with only one kind of optical label, a first optical label is associated with a first target, a second optical is associated with a second target, the first and second optical labels have overlapped spectra in a first and a second color channel; wherein the first and second target, upon excitation, are associated with different ratios of signal intensities from two color channels. In some embodiments, a third optical label is further associated with a third target in the same sample, the third optical label has overlapped spectra with the first and second optical label in the same combination of the first and second color channels, wherein the first, second and third target, upon excitation, are associated with three different ratios of signal intensities from these two color channels.

In some embodiments, each probe of a plurality of imaging probes is attached with two different optical labels of partially or completely overlapped emission spectra, such as two labels with their peak emission wavelengths separated by <100 nm, <50 nm, or <20 nm. In some embodiments, the fluorescence of two or more than two optical labels with close emission wavelengths passes through the same color channel for imaging. In some embodiments, the fluorescence of two or more than two optical labels with overlapped emission spectra passes through at least two color channels sequentially or simultaneously for imaging.

In some embodiments, each probe of a first plurality of imaging probes is attached with a first optical label only, each probe of a second plurality of imaging probes is attached with a second optical label only. The first and second optical labels here have partially or completely overlapped excitation or emission spectra, such as two labels with their peak emission wavelengths separated by <100 nm, <50 nm, or <20 nm. Each kind of optical label is detected by at least two color channels to form an intensity ratio, Different optical labels are detected by the same combination of color channels to generate different intensity ratios.

1. Labeling Scheme-1 and Labeling Scheme-2

The intensity levels of Labeling Scheme-1 and Scheme-2 for a target in a color channel are both determined by the total number of dyes associated with the target in that color channel. Different dyes are matched with different color channels. However, in Labeling Scheme-1, the number of each dye attached on a target is proportional to the number of target-binding probes (i.e., primary probes) associated with this dye on this target. Therefore, for each color channel and a chosen dye, the number of target-binding probes (or primary probes) associated with the corresponding dye defines the intensity level, but the number of the dye associated with a primary probe doesn't define the intensity level. In FIG. 2, panel a illustrates the simplest way of implementing the Labeling Scheme-1: direct labeling. Basically, nucleic acids or other kinds of targets are attached with imaging probes directly without any other intermediate hybridization based probes. In panel a, each target is bound to a plurality of probes each of which is attached with an optical label. The label is referred to as a reporter label. If a dye is used, the dye is referred to as a “reporter dye”. Each probe is attached with one reporter label only. Alternatively, each probe can conjugate multiple of the same type of dye as long as the number of dye per probe remains the same for each type of dye. From the five targets on the top to the bottom in FIG. 2a, the intensity ratio codes between the number of probes associated with Cy3 dyes and the number of probes associated with Cy5 dyes are 2:0, 0:2, 1:1, 1:2 and 2:1, respectively.

FIG. 2b illustrates the indirect labeling approach to implement Labeling Scheme-1. Basically, nucleic acids or other kinds of targets are attached with primary probes first and then primary probes bind with imaging probes.

FIG. 2c illustrates the indirect labeling together with branched DNA amplification approach to implement Labeling Scheme-1. Basically, nucleic acids or other kinds of targets are attached with primary probes first, and then primary probes bind with 2 tiers of signal amplification probes (first and secondary amplification probes), finally the secondary amplification probes bind with imaging probes. If necessary, more tiers of amplification probes can be used.

Labeling Scheme-2 can achieve these same ratios in a different manner. In contrast to Labeling Scheme-1, the intensity level is only proportional to the number of dyes used for each color channel but not related with the number of target-binding probes or primary probes per target. Different primary probes are associated with the same number and kinds of dyes. The panel a of FIG. 3 shows the simplest way to implement Labeling Scheme-2: direct labeling without any intermediate probes. Each target is bound to a number of probes with identical sets of optical labels for different probes on the same target. Various intensity level and ratio codes are achieved by changing the number and kind of optical labels on each probe. For instance, on the first target, each probe is conjugated to two Cy3 but no Cy5, corresponding to an intensity code of 2:0. On the fifth target, each probe is conjugated to two Cy3 and one Cy5, corresponding to an intensity code of 2:1.

FIG. 3b illustrates the indirect labeling approach to implement Labeling Scheme-2. Basically, nucleic acids or other kinds of targets are attached with primary probes first and then primary probes bind with imaging probes.

FIG. 3c illustrates the indirect labeling together with branched DNA amplification approach to implement Labeling Scheme-2. Basically, nucleic acids or other kinds of targets are attached with primary probes first, and then primary probes bind with 2 tiers of signal amplification probes (first and secondary amplification probes), finally the secondary amplification probes bind with imaging probes. If necessary, more tiers of amplification probes can be used.

The relative intensity and ratio of the optical labels can be adjusted easily to meet a requirement. For example, to achieve an intensity ratio of 1:2 (red: green color), one can mix 10 red dye with 20 green dye. To achieve a different ratio, one can mix 10 red dye with 15 green dye. However, as will be further described below, a color/intensity gap can be intentionally designed between different color/intensity codes to reduce or prevent incorrect detection due to intensity variation caused by optical labels, and binding variation of probes across different targets and locations, and other experimental conditions.

The implementation schemes illustrated in FIGS. 2 and 3 may have different advantages. For instance, the scheme illustrated in FIG. 2 is simpler in terms of probe design or synthesis especially for the direct and indirect labeling approaches illustrated in FIGS. 2a and 2b. However, if primary probes are bound to the target at different affinity or efficiency, the lack of uniformity in binding may result in labeling of a target with a color or intensity that is different from the design. Such a difference may not matter much for the first target in FIG. 2a, which is only bound to probes with one optical label. It could, however, have a more significant impact on the third target which should have a 1:1 ratio by design. If one of the Cy5-labeled probes fails to bind to the target, the resulting ratio would be 2:1, instead of 1:1. Labeling Scheme-2 illustrated in FIG. 3 can avoid such an issue, as each probe that binds to a target has the same combinations of dyes. Therefore, even if some of the probes fail to bind to the target, the impact on the resulting labeling may not be significant. At least the color ratio would be constant. For instance, in the last sample of FIG. 3a, if one probe fails to bind to the target, the ratio would still be 2:1.

It is contemplated that the Labeling Scheme-1 illustrated in FIG. 2 may be more suitable for FISH of long DNA or RNA sequences, where a number of primary probes can be used for detection and the binding variation among different binding regions doesn't affect the intensity variation of a number of probes for a target. The Labeling Scheme-2 illustrated in FIG. 3, on the other hand, may be more suitable for short DNA or RNA sequences, where only a limited number of primary probes can be used for detection and the binding variation among different binding regions may affect the intensity variation of a probe set for a target significantly.

The Label Scheme-1 and -2 can be used together to label different targets in a sample. For instance, for an RNA panel consisting of gene transcripts of different lengths, the longer transcripts may be labeled with the label coding scheme illustrated in FIG. 2 and the shorter transcripts may be labeled with the label coding scheme illustrated in FIG. 3.

2. Labeling Scheme-3

Yet another way of achieving high capacity detection employs different optical labels with overlapping color spectra so that multiple intensity levels can be obtained with the same color channel. This is illustrated in FIG. 4, panel a-c, and is referred to as an “Labeling Scheme-3.” In this scheme, a dimmer optical label (e.g., Cy3, which has yellow fluorescent color) is used along with a brighter label having a similar color or overlapping spectra (e.g., Alexa 546, which has an even brighter yellow fluorescent color). Typically, their peak emission wavelength is separated by <100 nm. Alternatively, they are separated by <50 nm, <40 nm, <20 nm, <10 nm, or <5 nm.

FIG. 4a illustrates the direct labeling approach to implement Labeling Scheme-3. Basically, nucleic acids or other kinds of targets are attached with imaging probes directly without any other intermediate hybridization based probes.

FIG. 4b illustrates the indirect labeling approach to implement Labeling Scheme-3. Basically, nucleic acids or other kinds of targets are attached with primary probes first and then primary probes bind with imaging probes.

FIG. 4c illustrates the indirect labeling together with branched DNA amplification approach to implement Labeling Scheme-3. Basically, nucleic acids or other kinds of targets are attached with primary probes first, and then primary probes bind with 2 tiers of signal amplification probes (first and secondary amplification probes), finally the secondary amplification probes bind with imaging probes. If necessary, more tiers of amplification probes can be used.

The colors of Cy3 and Alexa 546 are close or overlap in terms of emission spectra. They can be difficult to distinguish if used alone and recorded with the same color channel. However, when they are used in combination with 1-2 other optical labels from another color channel, their differences are enhanced and become distinguishable. By matching these dyes with the right color channel combination (e.g. one channel with an emission filter of 670-720 nm and the other channel with an emission filter of 580-620 nm)), different intensity ratios across two channels can be achieved (FIGS. 1b and 4). For instance, in FIG. 4a, on the fourth target, the brighter Cy5 is combined with the dimmer Cy3, and on the fifth target, the dimmer Cy5.5 is combined with the brighter Alexa546. Using one color channel to detect Cy5 and Cy5.5, the other color channel to detect Cy3 and Alexa 546, these two labeled targets can be differentiated by the relative intensity ratios between two color channels. Typically, 3-4 dyes are used to form two pairs of dyes to label two different targets, such as Cy5-Cy3 and Cy5-Alexa546, or Cy5-Cy3 and Cy5.5-Cy3. Different dye pairs can be imaged by the same two color channels to generate different intensity ratios.

Accordingly, in some embodiments, provided is a sample prepared for examination, comprising: a first plurality of probes bound, directly or indirectly, to a first targeted biomolecule in a biological sample, and a second plurality of probes bound, directly or indirectly, to a second targeted biomolecule in the biological sample, wherein each of the probes is attached with one or more optical labels such that: (a) the first target is associated with a first optical label, the second target is associated with a second optical label, and (b) these two different types of optical labels have the same or overlapping color spectra but have different intensities. In some embodiments, these two different types of optical labels may have different intensities in a color channel that differ by at least 2 fold, 2.5, 3, 4, 4.3 or 5 fold. In some embodiments, the different intensities of the two different types of optical labels differ by at most 10 fold, 8 fold, 7.5 fold, 6.3 fold, 5 fold, or 4 fold.

In some embodiments, the first and second targets are both further associated with a third optical label. Detecting the first and third optical labels associated with the first target, a first intensity ratio for the first target is generated. Detecting the second and third optical labels associated with the second target, a second intensity ratio for the second target is generated. Comparing the first and second intensity ratios, the first and second targets are differentiated.

Also provided is a probe set comprising a first probe and a second probe, wherein the first probe and the second probe are respectively attached with a first and second optical labels having the same or overlapping color spectra but different intensities, wherein the first probe and the second probe each is further attached with a third optical label, and wherein the first probe and the second probe are distinguishable optically. These probes may have different binding specificities, useful for targeting different target molecules.

When detecting the different probes, in some embodiments, the optically distinguishable probes, by virtue of the difference in intensity, can be detected by comparing the intensity ratio in at least two color channels.

Yet in another embodiment, provided is a sample prepared for examination, comprising: a first plurality of probes bound, directly or indirectly, to a first targeted biomolecule in a biological sample, and a second plurality of probes bound, directly or indirectly, to a second targeted biomolecule in the biological sample, wherein each of the probes is attached with one or more optical labels such that: (a) the first target is associated with a first optical label, the second target is associated with a second optical label, (b) these two different types of optical labels have the same or overlapping color spectra but have different intensities, and (c) the first target is further associated with a third optical label, the second target is further associated with a fourth optical label, and (d) the third and fourth optical labels have the same or overlapping color spectra but have different intensities. Detecting the first and third optical labels associated with the first target, a first intensity ratio for the first target is generated. Detecting the second and fourth optical labels associated with the second target, a second intensity ratio for the second target is generated. Comparing the first and second intensity ratios, the first and second targets are differentiated.

Also provided is a probe set comprising a first probe and a second probe, wherein the first probe and the second probe are respectively attached with a first and second optical labels having the same or overlapping color spectra but different intensities, wherein the first probe and the second probe are respectively attached with a third and fourth optical labels having the same or overlapping color spectra but different intensities, and wherein the first probe and the second probe are distinguishable optically. These probes may have different binding specificities, useful for targeting different target molecules.

3. Indirect Labeling and Signal Amplification

When using the Labeling Scheme 1-3, a target, in some embodiments, is directly bound to a probe that is attached with an optical label, as illustrated in the panel a of FIG. 2-4. In an alternative design, as illustrated in the panel b of FIG. 2-4, the optical label can be bound to the target indirectly with intermediate probes. In FIGS. 2b, 3b and 4b, primary probes (intermediate probes) without optical labels are bound to targets first, secondary probes with optical labels are bound to primary probes to achieve multiple intensity levels and intensity codes. In some embodiments, the optical label is attached to the probe after the probe has been already bound to the target.

In some embodiments, as illustrated in the panel b of FIG. 3, the primary probes consist of hybridization sequences and readout sequences. Hybridization sequences are complementary with the targeted sequences on DNA and RNA. Readout sequences are used to bind with dye labeled secondary probes. One primary probe can attach with up to 2 readout probes, one at 5′ and the other at 3′ end. By labeling different number of dyes and multiple different kinds of dyes, different intensity codes can be achieved.

In some embodiments, dyes are attached to oligo probes through cross-linking chemistry. Cross-linkers such as disulfide linkage, azide or alkyne groups are linked to oligo probes to facilitate either reversible or irreversible site-specific dye-oligo conjugation;

In some embodiments, intermediate probes are added after primary probe associated with a target molecule through signal amplification technology. In some embodiments, more than one tier of intermediate probes are added in-between the binding of primary probes and imaging probes. In some embodiments, primary amplifiers and secondary amplifiers are added in-between the binding of primary probes and imaging probes.

In some embodiments, branched DNA amplification technology is used for single molecule intensity coding, as illustrated in the panel c of FIG. 2-4. In the panel c, not just primary and secondary probes (or named as primary amplifiers), tertiary probes (or named as secondary amplifiers) are used to label one target with more optical labels so that higher signal intensity can be achieved. Other signal amplification technologies can also be used, such as hybridization chain reaction (HCR), signal amplification by exchange reaction (SABER), etc.

In some embodiments, signal intensities are amplified by balanced or unbalanced branched DNA amplification. As each primary probe (i.e. direct target binding probe) can be associated with amplifiers at both 5′ and 3′ end. For balanced branched DNA amplification, each primary is associated with the same number of dyes at each end of the primary probe as shown for the third target (labeled in 2:2) in FIG. 3c. For unbalanced branched DNA amplification, each primary probe is associated with different number of dyes at 5′ and 3′ end of the primary probe as shown for the fourth target and fifth target (labeled in 1:2 and 2:1) in FIG. 3c.

In some embodiments, a primary probe is associated with amplifiers at both 5′ and 3′ end, and the primary probe is associated with different kinds of dyes at 5′ and 3′ end. In some embodiments, a primary probe is associated with amplifiers at both 5′ and 3′ end, and the primary probe is associated with the same kinds of dyes at 5′ and 3′ end.

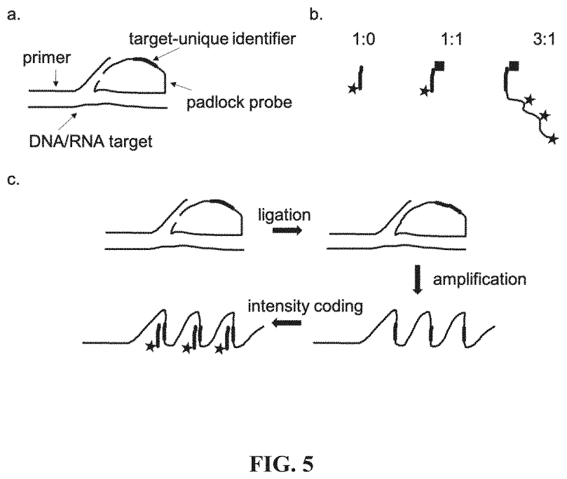

In some embodiments, enzymatic amplification methods are used to add intermediate probes in-between the binding of primary probes and imaging probes. One example of enzymatic amplification is rolling cycle amplification (RCA). In FIG. 5, target-unique identification probes are attached to targets first, and then a signal amplification step by rolling cycle amplification (RCA) is added to amplify target-unique identifiers, and finally the amplified probes with identification information of different targets are associated with imaging probes. This can be used for both nucleic acid and non-nucleic acid labeling.

4. Alternative Labeling Schemes for the Labeling Scheme 1-3

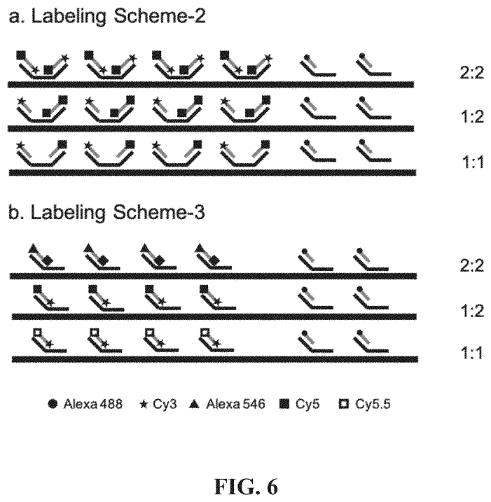

In some embodiments, other than using reporter dyes for intensity coding, a reference dye is added for labeling. This approach can be applied to any of the disclosed labeling schemes described herein. The reference dye is not used for creating intensity codes. Instead, it is used to differentiate non-specific binding from specific binding. This reference dye is imaged by a separate color channel from the color channels for intensity coding for error correction of non-specific binding signal. By doing colocalization analysis of the intensity coded reporter dyes and the reference dye, FISH dots of non-specific binding which don't have signal in the channel for the reference dye can be removed. In some embodiments, a reference dye associated with an additional plurality of primary probes is added to label each target (FIG. 6).

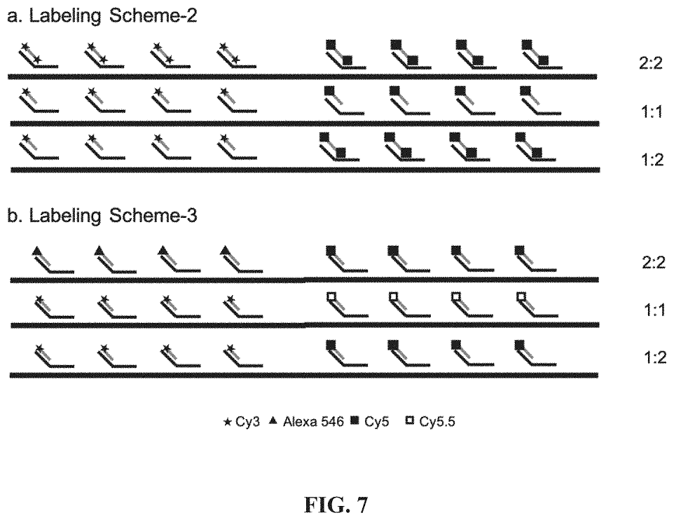

In some embodiments, when using Labeling Scheme-2 and Labeling Scheme-3, a differentiated labeling approach is used. In other words, different optical labels are associated with different sets of primary probes for the same target (FIG. 7).

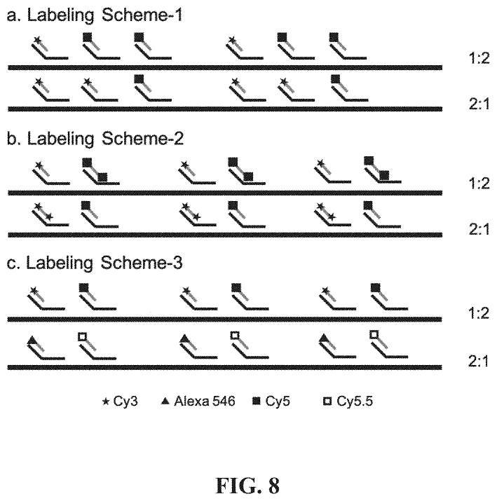

In some embodiments, different primary probes associated with different optical labels for a target bind with the target in an alternating order. This approach can minimize the influence of the variation of the binding affinity of various primary probes among different targeted regions to the intensity variation of its assigned intensity code. Take Labeling Scheme-2 as an example, as illustrated in FIG. 8b, for the first RNA target with an intensity code of 1:2 (the two color channels follow the order of Cy3 and Cy5), every two primary probes form a group along the RNA target. Each group is comprised of one primary probe associated with one Cy3 dye and one primary probe associated with two Cy5 dyes. For the second RNA target with an intensity code of 2:1 in FIG. 8b, every two primary probes form a group along the RNA target as well. Each group is comprised of one primary probe associated with two Cy3 dyes, one primary probe associated with one Cy5 dye,

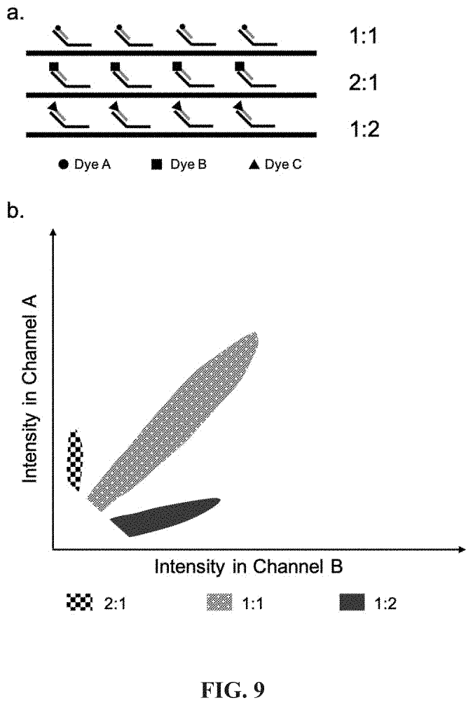

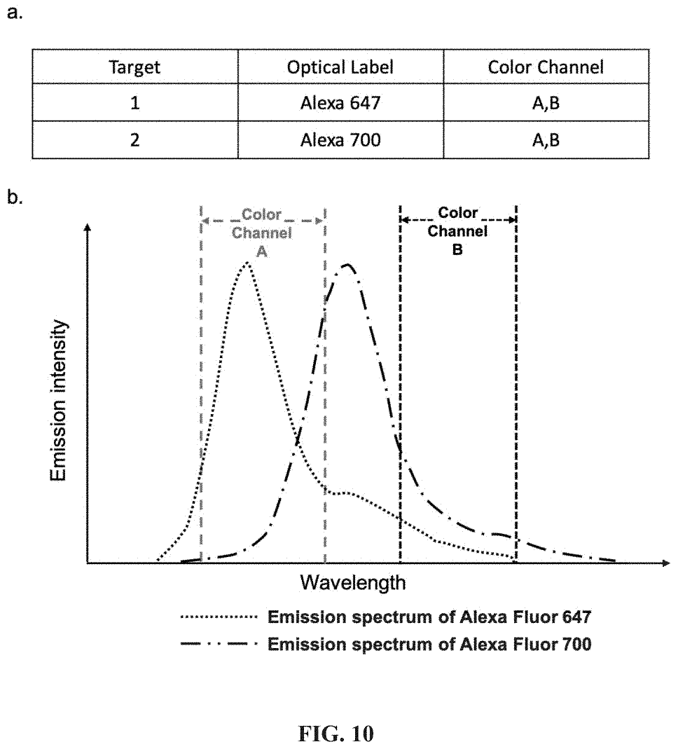

In some embodiments, the Alternative Labeling Scheme-3 as illustrated in FIG. 9 is used. In this Alternative Labeling Scheme-3, only one optical label is used to label a target (FIG. 9). Different targets are labeled with different optical labels with overlapped excitation or emission spectra in the same combination of color channels (at least two). For example, Alexa 647 and Alexa 700 can be used for this scheme (FIG. 10, Example 10). Each optical label is imaged by the same combination of color channels (at least two color channels) and thereof different optical labels generate different intensity ratios by comparing the intensity of the label in these channels. Typically, Optical labels are attached on oligonucleotide or peptide probes for labeling, such as single stranded DNA oligos or PNA.

In some embodiments, indirect labeling is used to achieve the Alternative Labeling Scheme-3. In some embodiments, indirect labeling and signal amplification are combined to achieve the Alternative Labeling Scheme-3. In some embodiments, indirect labeling and branched DNA signal amplification are combined to achieve the Alternative Labeling Scheme-3. In some embodiments, to achieve the Alternative Labeling Scheme-3, one primary probe and one imaging probe are used to detect a target. In some embodiments, at least 2 or 4 primary probes are associated with a first target and a first optical label. In some embodiments, at least 12 primary probes are associated with the first target and the first optical label. In some embodiments, at least 24 primary probes are associated with the first target.

In some embodiments, the Alternative Labeling Scheme-3 is combined with the Labeling Scheme-2 or Scheme-1 to detect at least two targets. For example, to detect target A and B, each copy of target A is labeled by one Dye A, and each copy of target B is labeled by 20 Dye B. These two dyes are detected by two color channels and generated two different intensity ratios for these two targets. Target A is encoded by intensity code of 2:1, and target B is encoded by the intensity code of 1:2.

In some embodiments, a biological sample for examination comprises a first plurality of probes bound, directly or indirectly, to a first target molecule, cell or tissue in a biological sample, and a second plurality of probes bound, directly or indirectly, to a second target molecule, cell or tissue in the biological sample, wherein each target is associated with only one kind of optical label, a first optical label is associated with a first target, a second optical is associated with a second target, the first and second optical labels have overlapped spectra in a first and a second color channel; wherein the first and second target, upon excitation, are associated with different ratios of signal intensities from two color channels. In some embodiments, in the same sample, the first and second optical labels have overlapped spectra in a third color channel, wherein the first and second target, upon excitation, are associated with different ratios of signal intensities from three color channels. In some embodiments, in the same sample, the ratio of the number of the first optical label associated with the first target and the number of the second optical label associated with the second target is at least 2. In some embodiments, the ratio of the number of the first optical label associated with the first target and the number of the second optical label associated with the second target is at least 4, 4.5, 6, 7.2, 8, or 10.

5. Sparse Coding

In some embodiments, to reduce the overlap rate (the error rate of species assignment) and control the sparsity of intensity coding, an error-robust subset of possible codes should be chosen for practical applications. For example, in the case of N=2 and M=2, eliminating the codes of 2:0, 0:2, 1:1 and 2:2 may reduce the intensity overlap to <5% while still retaining 4 different intensity combinations (1:0, 0:1, 1:2, 2:1) with only 2 color channels (see Example-1).

In some embodiments, when different intensities of an optical label are employed by different probes, it can be useful to ensure that the difference in neighboring intensity levels is above a threshold to reduce incorrect detection when an intensity level shifts lower or higher. The incorrect detection may be a result of limited detection capability, chromatic aberrations, different probe binding efficiencies, the intensity variation of optical labels, or because different probes have different accessibility to their targets, without limitation. For instance, a target DNA that is bound to 5 Cy3 dyes and 1 Cy5 dye may be easily distinguishable from another target DNA that is bound to 1 Cy3 dyes and 1 Cy5 dye, but may not be easily distinguishable from yet another target DNA that is bound to 4 Cy3 dyes and 1 Cy5 dye.

In some embodiments, the ratio of a first optical label associated with a first target and the first optical label associated with a second target differ by at least 2, 2.5, 3, 3.4, 4, or 5 fold. In some embodiments, the intensity ratio of a first optical label associated with a first target and the first optical label associated with a second target differ by at least 2, 2.5, 3, 3.4, 4, or 5 fold.

In some embodiments, the ratio of a first optical label associated with a first target and the first optical label associated with a second target differ by no more than 100 fold. In some embodiments, the ratio of a first optical label associated with a first target and the first optical label associated with a second target differ by no more than 50 fold. In some embodiments, the ratio of a first optical label associated with a first target and the first optical label associated with a second target differ by no more than 10 fold.

In some embodiments, the number of a first optical label associated with a first target and the number of the first optical label associated with a second target differ by at least 2. In other words, no two different target molecules will differ by just one optical label when both are bound to the same optical label, and the same number and type of other optical labels. One particular example of such an arrangement is that only odd numbers of a particular optical label is used in a biological sample. In another example, only even numbers of an optical label is used.

In some embodiments, the number of a first optical label associated with a first target and the number of the first optical label associated with a second target differ by at least 10, 20, 40, 100, 500, or 1000, without limitation.

In some embodiments, the number of a first optical label associated with a first target and the number of the first optical label associated with a second target differ by less than 1000, 980, 900, 800, 750, 620, 500, 400, 300, 200, 120, or any other integer number that is <1000. In some embodiments, the number of a first optical label associated with a first target and the number of the first optical label associated with a second target differ by less than 100, 95, 80, 70, 65, 50, or any other integer number that is <100.

In some embodiments, when using the Labeling Scheme-3, two optical labels with their peak emission wavelength separated by <100 nm are associated with two different targets and detected with the same color channel. However, to decrease spectrum overlap and increase coding sparsity, the peak emission wavelength of the first optical label is separated from the peak emission wavelength of the second optical label by >5 nm. Alternatively, they are separated by >10 nm, >15 nm, >18 nm, or >20 nm.

In some embodiments, the intensity differences (defined by the peak, mean or median intensity of the intensity distribution histogram) between any two intensity levels (excluding 0) used for an intensity coding scheme are separated by at least a factor of 2, 2.5, 3, 3.4, 4, 4.6, or 5. For example, for a coding scheme of M=3, the intensity of Level 2 is 2× of that of Level 1. The intensity of Level 3 is 2× of that of Level 2, 4× of that of Level 1. In some embodiments, the intensity differences between any two intensity levels (excluding 0) used for an intensity coding scheme are separated by at least a factor of 3. For example, for a coding scheme of M=3, the intensity of Level 2 is 3× of that of Level 1. The intensity of Level 3 is 3× of that of Level 2, 9× of that of Level 1. The difference between two intensity levels is not limited to a specific factor. The factor difference is also not limited to integers and can be fractional such as 3.5×, 5.2×, or 10.6×.

6. Combing Multiple Labeling Schemes

In some embodiments, multiple labeling schemes are combined to increase the intensity gap of adjacent intensity codes and detection accuracy. In some embodiments, multiple labeling schemes are used to label different targets in a target panel at the same time. For example, using Labeling Scheme-1 to detect a first target in a panel, Labeling Scheme-3 to detect a second target in the same panel. In some embodiments, multiple labeling schemes are combined to label the same target. For example, combining Labeling Scheme-2 and Scheme-3 to detect a first target in a panel, and combining Labeling Scheme-1 and Scheme-3 to detect a second target in the same panel.

In some embodiments, the Labeling Scheme-2 and Scheme-3 are combined to get better separation of two intensity codes. For example, instead of just using the dye pairs of Cy5-Cy3 (1 Cy5 dye and 1 Cy3 dye per primary probe) and Cy5.5-Alexa 546 (1 Cy5.5 dye and 1 Alexa546 dye per primary probe) to label two targets, 2Cy5-Cy3 (2 Cy5 dye and 1 Cy3 dye per primary probe) can be used to label the first target, Cy5.5-2Alexa546 (1 Cy5.5 dye and 2 Alexa546 dye per primary probe) can be used to label the second target.

In some embodiments, differentiated labeling and the Labeling Scheme-3 are combined to detect at least 2 targets. For example, 4 dyes with overlapped spectra (Cy5, Cy5.5 have overlapped emission spectra in color channel-1; Cy3, Alexa 546 have overlapped emission spectra in color channel-2) are used to detect 2 targets A and B. Cy5 and Cy3 are used together to detect target A but these two dyes are associated with 2 different sets of primary probes. Cy3 and Alexa 546 are used to detect target B but these two dyes are associated with 2 different sets of primary probes.

In some embodiments, unnatural nucleic acids and non-nucleic acid based molecules such as LNA, PNA and xenonucleic acids (XNA) which have higher binding affinity towards natural nucleic acids than that between natural nucleic acids and themselves can be used for different kinds of probes, such as primary probes, amplification probes, imaging probes. Such uses can increase the genomic resolution for a DNA/RNA target. In other words, more probes with a shorter sequence per probe can be designed with this approach than that using natural oligonucleotides for the same target. This strategy applies to all labeling schemes above.

In some embodiments, a hybrid of peptide and oligonucleotide sequences are used as probes, such as a hybrid of PNA and natural nucleotides.

7. Labeling Scheme-4

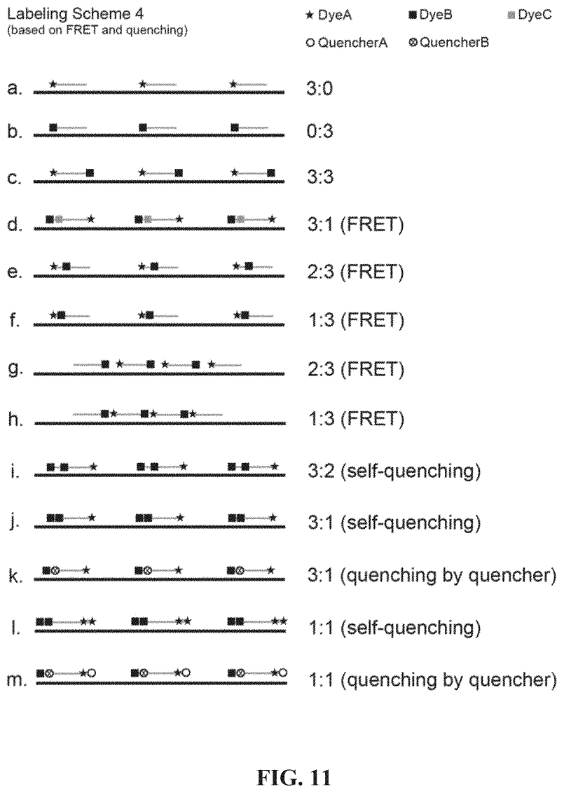

Yet another way to adjust intensity level and distribution at single molecule level is through fluorescence enhancement, or fluorescence quench. This is named as Labeling Scheme-4. This scheme can be used to adjust intensity variation or create a new intensity level. Fluorescence enhancement or quenching can be generated by conjugating a reporter dye with an intensity enhancing molecule or quenching chemical group. As illustrated in FIG. 11, multiple ways can be utilized to induce fluorescence quench, such as associating a dye with a quenching chemical group like BHQ (black hole quencher dye), associating a dye with another dye to create a FRET dye pair (such as linking Cy3 and Cy5 together), shortening the distance of adjacent dyes along the targeted nucleic acid sequence.

In some embodiments, at least one optical label associated with a target is attached with more of the same optical label. In some embodiments, at least one optical label associated with a target is attached with a different optical label which can quench the optical label by FRET. In some embodiments, at least one optical label associated with a target is attached with a quencher chemical group like BHQ. In some embodiments, at least two of the same optical labels associated with a target are separated by no more than 10 nucleotides.

Increasing the labeling density of an oligo probe can induce optical quenching. For example, associating an oligo of 100 nt with less than 5 optical labels may not induce quenching but increasing the optical labels to more than 5 optical labels will induce fluorophore quenching. In some embodiments, an oligo probe is associated with multiple optical labels so that every 100 nt of the probe sequence are associated with more than 5 optical labels. In some embodiments, an oligo probe is associated with multiple optical labels so that every 100 nt of the probe sequence are associated with more than 6, 8, 10, or 15 optical labels. In some embodiments, an oligo probe is associated with multiple optical labels so that every 100 nt of the probe sequence are associated with more than 50, 60, 80, or 100 optical labels.

In some embodiments, the Labeling Scheme-4 is combined with other labeling schemes to label a target. In some embodiments, the Labeling Scheme-4 and 2 are combined to detect a target. In some embodiments, the Labeling Scheme-2, 3 and 4 are combined to detect a target.

8. High Capacity Detection of Non-Nucleic Acid-Based Targets

High Capacity In-situ Biomolecule Detection: The intensity coding technology developed for in-situ RNA and DNA detection can be expanded to other biomolecule in-situ detection such as protein, lipid, sugar molecule, epigenomic modification and epitranscriptomic modification, etc. The general labeling scenario is attaching non-nucleic acid targets with target-specific oligonucleotide or peptide sequences (such as PNA) first to assign intensity barcodes to the targets, and then associating intensity coded detection probes with the target-specific oligos or peptides by hybridization. The target-specific oligonucleotides or peptides can be attached with targeted molecules through various kind of linkers or intermediate molecules.

In some embodiments, different targets are associated with different kinds of primary probes. In some embodiments, different targets are associated with different number and kinds of primary probes. In some embodiments, at least a first target is associated with two or more than two primary probes. In some embodiments, the first target is associated with two or more than two of the same primary probes. In some embodiments, the first target is associated with two or more than two of different primary probes.

9. High Capacity In-Situ Protein Detection

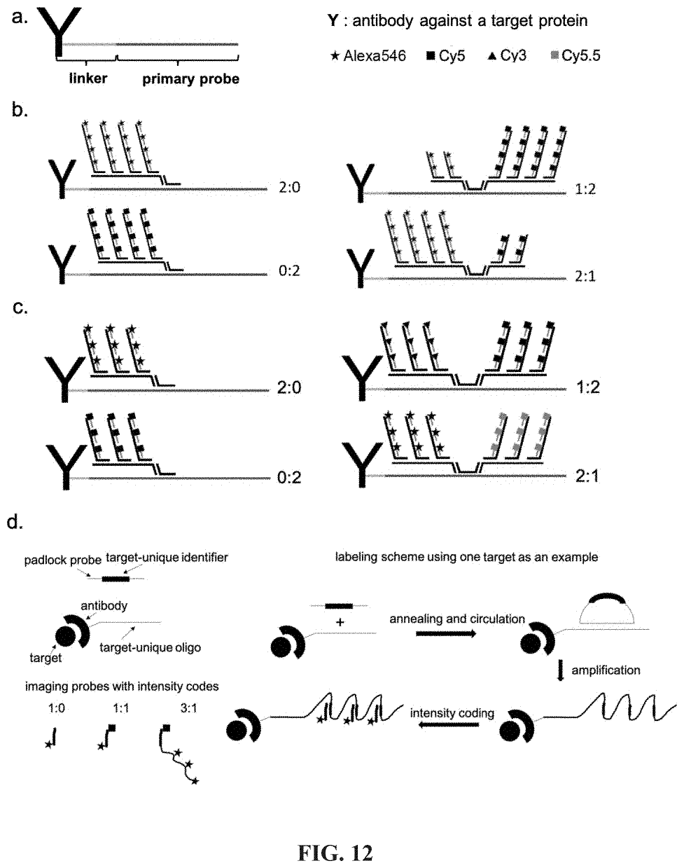

A labeling scheme for labeling protein targets is illustrated in FIG. 12 and described below. In one embodiment, proteins are recognized by primary antibodies. Primary antibodies are attached with target-specific primary oligonucleotides or peptide nucleic acids (PNA). Different kinds of proteins are associated with different target-specific primary oligonucleotides or peptide nucleic acids. Then optical probes can be added to recognize different target-specific primary probes to achieve different intensity ratio codes. Signal amplification technology such as rolling cycle amplification, branch DNA amplification can be further added on top of the oligo and antibody binding chemistry to detect individual proteins so that more intensity levels and a variety of intensity ratio codes can be achieved.

In some embodiments, proteins can be attached with small or big molecules such as biotin, GFP, HA-tag, SNAP-tag, Halo-tag to facilitate site-specific labeling. Different proteins have different epitopes which are recognized by different antibodies. Different antibodies are attached with different target-specific primary probes. Different primary probes are detected by different optical probes to achieve different intensity ratio codes.

In some embodiments, different protein targets are associated with different kinds of primary probes. In some embodiments, different protein targets are associated with different number of primary probes. In some embodiments, at least a first protein target is associated with two or more than two primary probes. In some embodiments, the first protein target is associated with two or more than two of the same primary probes. In some embodiments, the first protein target is associated with two or more than two of different primary probes. 10. High Capacity In-Situ Detection of Epigenetic Modifications

Epigenetic modifications refer to functionally relevant changes to the genome that do not involve a change in the nucleotide sequence, such as DNA or RNA methylation, histone modification. To do intensity coding, these modifications can be first detected with oligo attached antibodies or any oligo-attached molecules that recognize and bind to the epigenetic modifications. Then these oligos can work as primary probes to further hybridize with different intensity coded probes to achieve various intensity ratio codes. Typically, signal amplification technology such as branched DNA amplification, or rolling cycle amplification are combined with oligo labeled antibodies to achieve various intensity levels and intensity ratio codes.

11. High Capacity Cell Detection

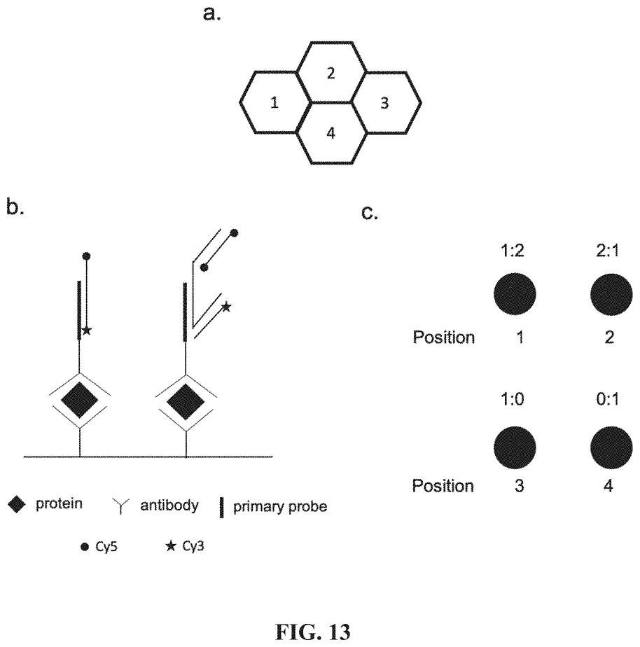

High capacity detection by intensity ratio coding can be used to detect, differentiate and sort multiple cell populations with limited number of colors and limited kinds of optical labels. The scenario is labeling different cell types through cell type-specific biomarkers such as RNA and protein (FIG. 13a). Different cell markers are associated with different intensity coded optical probes. For example, intensity ratio coding developed here can be used to detect 4 proteins CD34, CD45, CD9, Pancytokeratin together. Each protein of these 4 is a cell marker. Therefore, at least 4 cell types can be distinguished by intensity barcoding of these 4 proteins.

12. High Capacity In-Vitro Molecule or Particle Detection

High capacity detection by intensity ratio coding can be used to detect and differentiate single molecules or single particles in vitro. For example, as illustrated in FIG. 13b, individual proteins can be captured onto glass substrate randomly and sparsely, and detected by oligo probes with different intensity ratio codes. Alternatively, as illustrated in FIG. 13c, different kinds of molecules can be captured on a substrate and spatially separated with each other in an ordered pattern. Then different group of molecules at different locations can be detected by intensity coded optical probes.

D. Optical Setup

Optical setup: For intensity coded sample imaging, at least two kinds of instruments can be used: microscope and flow cytometry. For microscope imaging, biological samples are typically attached onto a glass scaffold and imaged by a microscope with a CCD or camera. For the best performance of intensity imaging through microscopy, a laser excited fluorescence microscope such as a spinning disk confocal microscope and a highly sensitive camera such as EMCCD or sCMOS camera are required to achieve high quality images with the best signal to background ratio and signal to noise ratio. For flow cytometry, samples are labeled with intensity barcoded probes and then detected by PMT. Yet for some other samples, they are detected by other kinds instruments such as a plate reader.

E. Image Analysis: Normalization and Error Correction

1. Intensity Normalization

This step can be used to convert the intensity value of all color channels related with optical spots such as FISH spots into individual intensity ratios. Taking RNA FISH as an example, depending on the intensity codes used for RNA encoding, absolute intensity values of each color channel or relative intensity ratio among different color channels can be used to calculate the intensity ratio of FISH spots. For example, if using a 2 color coding scheme of 1:1 and 2:2 to label two RNA species, absolute intensity values need to be used to calculate the intensity ratio of each FISH spot (see Example 1). However, if only a portion of the codes from a whole code spectrum are used to minimize intensity overlap between any two intensity codes, one can use just the relative intensity among different channels (at least two color channels) to calculate the intensity ratio (see Example 1).

2. Code Assignment Using the Reference Intensity Code Map

As described above, use of sparse coding can help leaving suitable space between neighboring intensities, thereby reducing overlapping signals and reducing error. Nevertheless, in some embodiments, error correction can be employed to further ensure signal reading accuracy.

In some embodiments, error correction requires accurate measurement of intensity variation (or intensity ratio variation) of each intensity code and its corresponding probe design scheme. The intensity variation of each intensity code is determined by the intensity variation of the probe labeling scheme, which is related with multiple factors, such as the exact probe sequence design, the design of optical labels, the target of interest, the sample used, the staining and imaging condition. A reference map of the intensity variation of all intensity codes can be measured with reference samples. Based on such a reference intensity code map, the boundary of the intensity distribution of an intensity code and its labeling scheme can be determined (See Example 4). The overlap rates of any two intensity codes can be also accordingly determined.

A simple simulation of measuring the intensity variation and overlapping rates of intensity codes is shown in Example 1.

### Code Assignment and Spot Rejection

As illustrated in FIG. 1c, ideally, each FISH spot or intensity spot from optical images will be assigned to its nearest intensity code based on the distance of the measured intensity (or intensity ratio) of a spot to the centroid intensity (or intensity ratio) of a pre-determined intensity code in the intensity coordinate map. Spots with equal distances to two near intensity codes will be discarded.

The formula to calculate the intensity distance is as below:

D²=(x₁−x₀)²+(y₁−y₀)²+(z₁−z₀)²+ . . .

In the formula, D represents the distance of a measured intensity of an intensity spot from an experimental sample to the centroid intensity of a pre-determined intensity code. (x₁, y₁, z₁, . . . ) represents the measured intensity of an intensity spot in a specific color channel. (x₀, y₀, z₀, . . . ) represents the individual intensity of a per-determined intensity code in a specific color channel, which are named as grid intensity coordinates here. If an intensity coding scheme using more than 3 colors, additional variables beyond x, y and z can be added onto the right side of the formula above.

Take N=2 and M=2 (2 color and 2 intensity levels) as an example, if an intensity code has an intensity of 1:2 (The intensity in color channel-1 is 1, the intensity in color channel-2 is 2). x₀=1, y₀=2 represents the position of this code on the 2 color or 2 dimension intensity coordinate map. If a measured intensity from a FISH spot is 1:1.7, x₁=1, y₁=1.7. By calculating its distance to its surrounding 4 grid coordinates, one can assign this FISH spot to the intensity code of 1:2. If a measured intensity from a FISH spot is 1:1.5, x₁=1, y₁=1.5. By calculating its distance to its surrounding 4 grid coordinates, it has equal distance to the grid coordinates (1,1) and (1,2). This FISH spot is rejected due to the ambiguity of code assignment.

Practically, a variety of factors may change the intensity distribution of a probe labeling scheme. So the intensity distribution of a chosen probe labeling scheme and the corresponding intensity code may have an irregular boundary in the intensity coordinate map (FIG. 1d). In order to assign all intensity spots to the right intensity rode, the boundary of all intensity codes need to be determined experimentally using reference samples. By labeling a target of interest with the designed intensity code and labeling scheme in a reference sample that is similar to the experimental sample, the intensity distribution of each labeling scheme and its corresponding intensity code can be measured individually. Then based on the density distribution of all intensity spots generated from the reference sample, the boundary can be determined. Comparing the measured intensity of an intensity spot from an experimental sample with the boundary determined with the reference sample, if the intensity is located in the boundary of an intensity code, this spot is assigned with the corresponding intensity code, otherwise it is rejected; if the intensity is located in two boundaries of two intensity codes, this spot is rejected due to ambiguity of assignment.

One way to determine the boundary of an intensity code is measuring the intensity distance of any two intensity spots in the intensity coordinate map using the formula below:

D²=(x₁−x₂)²+(y₁−y₂)²+(z₁−z₂)²+ . . .

In the formula, D represents the distance of the measured intensity of a random intensity spot from a reference sample to the measured intensity of another random intensity spot from a reference sample. (x₁, y₁, z₁, . . . ) and (x₂, y₂, z₂, . . . ) represents the measured intensity of a random intensity spot in a specific color channel. If an intensity coding scheme using more than 3 colors, additional variables beyond x, y and z can be added onto the right side of the formula above. Intensity spots with intensity distance below a certain range can be grouped together to form a cluster of intensity spots and therefore generate the boundary of a cluster, i.e., an intensity code.

### Error Robust Intensity Coding

For intensity coding, an error correction scheme based on the distance of the experimental intensity ratio to the centroid position of the pre-determined intensity codes of a labeling scheme will be used (FIG. 1c). Specifically, for more robust detection, one may consider an experimental ratio has ambiguity if its distance difference to two adjacent grid coordinates is within a certain range, such as ±0.01 for two color intensity coding, and discard the corresponding FISH spot for code assignment.

Practically, an error correction scheme for intensity coding is generating a reference intensity code map based on the intensity distribution of intensity codes in reference samples (Example 4). Comparing the intensity of an intensity spot from an experimental sample with the intensity boundary of all intensity codes in the reference intensity code map, an intensity spot can be assigned with the right intensity code or rejected.

Another error correction is making use of the sparsity of the entire coding space. One major source of error in HC-smFISH and high capacity detection by intensity coding is the intensity overlap between two intensity codes, resulting in the mis-assignment of intensity code. To solve this error, one can just choose a portion of the intensity codes available so as to increase the intensity gap between two adjacent intensity codes and the sparsity of the codes used in the entire coding space.

F. Summary of the Imaging Scheme for Intensity Coding

In summary, this invention provided a method of using a limited number of color channels (this number is denoted as N) and a variety of optical labels (the number of different labels is denoted as L) to detect a lot of different targets (the number of different targets is denoted as P) which is more than the number of color channels available. Typically, the number of targets that is detected can be denoted in this formula: P≥2^{N} (N≥1). In some embodiments, we use more optical labels than the number of color channels to detect P kinds of targets (P>N, L>N), which is the case for Labeling Scheme-3. In some embodiments, we use the same number of optical labels as the number of color channels to detect P kinds of targets (P>N, L=N), which is the case for Labeling Scheme-1 and 2. In some embodiments, we use equal number or more of optical labels than the number of color channels to detect P kinds of targets (P>N, L≥N), which is the case for the Alternative Labeling Scheme-3. A hybridization-based probe assay, but not limited to hybridization-based probes, is typically used to implement various intensity coding schemes here.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Intensity Variation of Single Molecule RNA FISH and a Simulation of 2 color HC-smFISH

Single molecule RNA FISH: A Rapid RNA FISH using TFRC mRNA probes from LGC Biosearch Technologies was done in HeLa cells. The TFRC probes were comprised of 48 oligo probes. Each probe was 20 nt long and labeled with one Quasar 570 dye at 5′ end. 8-well glass bottom culture chambers (Ibidi) were used for sample imaging. Cells were fixed in 4% paraformaldehyde (PFA; Electron Microscopy Sciences) at room temperature for 5-10 min and then permeabilized in pure methanol (Sigma) for 5-10 min. Then the sample was processed by the following steps: (1) thorough heat denaturation in a dry bath in 80% formamide (Sigma) and 2×SSC buffer at 75° C. for 10 min; (2) addition of short DNA oligonucleotide probes with a hybridization time of 10 min in a hybridization buffer of 10% formamide, 20% dextran sulfate (Sigma, MW>500,000) and 2×SSC; (3) washing by 2×SSC buffer 2-3 times; (3) Imaging the sample. The probe concentration was 100 nM during hybridization.

Imaging setup: Fluorescence images were acquired on an inverted fluorescence microscope (Ti-E; Nikon, Melville, N.Y.) with a Plan apo VC 100× 1.45 N.A. oil immersion objective. Andor Borealis CSU-W1 spinning disk confocal was connected to the left port of the microscope body. 4 lasers (100 mW at 405, 561, and 640 nm; 150 mW at 488 nm) were equipped to generate fluorescence excitation and emission in DAPI, FITC, Cy3, and Cy5 channels. The internal dichroic in the CSU-W was used for the incoming lasers. No additional excitation filters were used for spinning disk confocal imaging. 4 lasers were used and matched with 4 emission filters (Chroma): 405 nm laser with an emission filter at 450/50 m; 488 nm laser with an emission filter at 525/50 m, 561 nm laser with an emission filter at 600/50 m; 640 nm laser with an emission filter at 700/75 m. All emission filters were mounted into a motorized filter-wheel (Lambda 10-B; Sutter Instrument, Novato, Calif.). A motorized microscope stage (Applied Scientific Instrumentation (ASI), Eugene, Oreg.) was used to control the xy and z translation of the sample. The fluorescent images were recorded with a sCMOS camera (Andor Zyla 4.2 sCMOS camera) with 200 ms exposure time. The microscope, light source, motorized stage, motorized filter wheel, and camera were controlled through custom configuration in the software Micro-Manager (www.micro-manager.org).

Image Analysis: Images were first treated with a real-space bandpass filter that suppresses pixel noise and long-wavelength image variations. The bandpassed image were then applied with an algorithm to find local maxima to pixel level accuracy which provided a rough guess of the centers of fluorescent spots. To distinguish the fluorescent spots from background signal arising from the non-specific binding probes, the intensity threshold was required to be set up in this step where we only picked up spots with intensity four times of the mean intensity of an area without fluorescent spots. The picked local maxima were then further fitted by a Gaussian-fitting algorithm to achieve intensity, spot number and sub-pixel resolution of locations of fluorescent spots, assuming that all reasonable fluorescent spots were confined within 10 pixels by 10 pixels region.

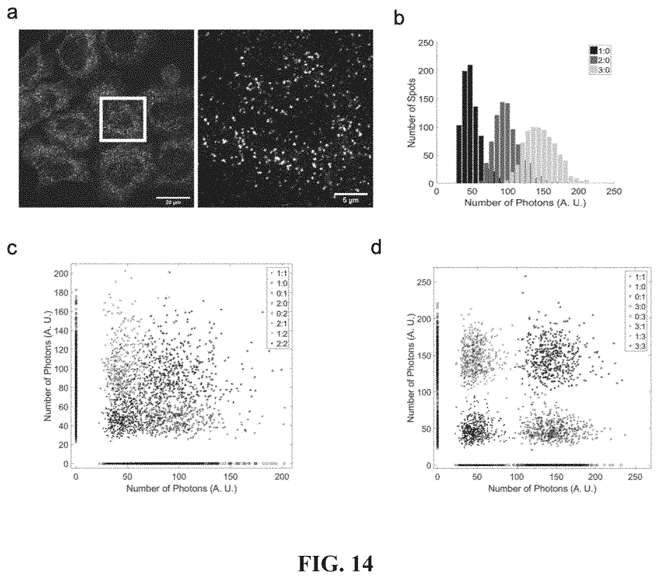

Results and Discussion: Ratio 1:0 is the experimental result from 790 spots detected by single Quasar-570 dye labeled TFRC probes. Ratio 2:0 and 3:0 are simulated data based on Ratio 1:0 with double intensity and triple intensity per spot, respectively. Ratio 2:0 has 40% spots overlapping with Ratio 1:0. Ratio 3:0 has <5% spots overlapping with Ratio 1:0. The intensity variation of Quasar-570 labeled TFRC spots shows that it is feasible to separate two ratios 1:0 and 3:0 very well with the spot overlapping or misidentification rate <5% (FIG. 14b).

The intensity distribution of single Quasar570 dye labeled oligo probes for TFRC transcripts (LGC Biosearch Technologies) were used for simulation here.

The simulation was done as follows:
(1) We first obtained the distribution of intensity (number of photons) from the experimental results of single dye labeled TFRC RNA molecules in Example-4 and did a histogram analysis;
(2) Then we fit a probability density function (PDF) of different number of photons according to the histogram;
(3) Based on the PDF, we could simulate the data sets for each ratio. Since the PDF gave the probabilities of all the values, our simulation generated random numbers according to their probabilities using the random number generator in Matlab (R2016a). For example, for 1:0, we just used the PDF to simulate 500 values, which gave the x values of 500 dots for the 1:0 data;
(4) To simulate 500 dots of the intensity ratio 1:1, we simulated 500 values from the PDF of step 2 to give x values of 500 dots then simulated another 500 values from the same PDF to give y values of 500 dots, since the x values and y values of the dots were independent and obeying the same PDF as assumed in our current simulation;
(5) To simulate 500 dots of the intensity ratio 1:2, we calculated the PDF for the higher intensity level (double intensity here) through the convolution of the PDF obtained from step 2 and the same PDF. Then as we did in step 4, we simulated the intensity ratio 1:2.
(6) To simulate 500 dots of the intensity ratio 1:3, we calculated the PDF for the higher intensity level (three dye here) through the convolution of the PDF from step 2 for single dye and the PDF from step 5 for two dyes. Then as we did in step 4, we simulated the intensity ratio 1:3.

FIG. 14c shows the simulation results of dots overlap with 2 color and 2 intensity levels (N=2 and M=2). Ratio 1:0, 2:0, 0:1, 0:2 can separate from the middle 4 intensity ratios very well because one color channel of the first 4 ratios has no signal. The overlapping rates among the middle 4 intensity ratios are listed in the table below. The overlapping rates of 1:1&2:2 and 1:2&2:1 are <10%, which are significantly smaller than that of other 4 combinations, which are between 10 and 70% as listed in the table below.

| Intensity Ratio | | Overlapping ratio of | Overlapping ratio |
|---|---|---|---|
| 1^{st} ratio | 2^{nd} ratio | the first probe | of the second probe |
| 1:1 | 2:1 | 14% | 71% |
| 1:1 | 1:2 | 16% | 68% |
| 1:1 | 2:2 | 7% | 9% |
| 2:1 | 2:2 | 15% | 62% |
| 1:2 | 2:2 | 15% | 73% |
| 1:2 | 2:1 | 3% | 5% |

FIG. 14d shows the simulation results of dots overlap with 2 color and 2 intensity levels but with a larger intensity gap between the first and second intensity levels. The second intensity level has on average 3× intensity of the first intensity level. The overlapping rates of any two intensity ratio codes are less than 1%, which means they are very well separated with each other as listed in the table below. If using these 8 codes for 8 RNA detection in the same round of FISH imaging, >99% FISH dots should be correctly assigned to the right RNA species.

| | | Percentage of the | Percentage of the |
|---|---|---|---|
| Intensity Ratio | | overlapped dots | overlapped dots |
| 1^{st} ratio | 2^{nd} ratio | in the 1^{st} ratio | in the 2^{nd} ratio |
| 1:1 | 3:1 | <1% | <1% |
| 1:1 | 1:3 | <1% | <1% |
| 1:1 | 3:3 | 0 | 0 |
| 3:1 | 3:3 | <1% | <1% |
| 1:3 | 3:3 | <1% | <1% |
| 1:3 | 3:1 | 0 | 0 |

Based on results above, to minimize the intensity overlap between any intensity codes, the intensity gap should be big enough. In this way, the single molecule detection efficiency can be maximized.

However, as the gene accessibility varies a lot among different DNA or RNA targets and different kinds of cell and tissue samples, the actual overlapped rates of intensity codes to be used for a DNA/RNA panel has to be validated by experiment for the best accurate code assignment and highest DNA/RNA detection efficiency.

### Example 2

### Generating Multiple Dye Labeled Oligo Probes

Multiple dyes of the same color or different colors can be conjugated to each oligo probe for multi-color intensity coding. For two-dye labeling per oligo, we can use short oligos (typically 20-50 nt) and terminal labeling at both ends of each oligonucleotide. For three- and four-dye labeling, we will use longer oligo probes but still <=100 nt to accommodate multiple dye conjugation per oligo. Each long probe consists of a hybridization sequence and a readout sequence. The hybridization sequence will be 20-40 nt long with natural nucleotides. The readout sequence will be typically at least 15 nt in length. The dye labeling density will be controlled at >10 nt per dye (usually ˜20 nt per dye) to minimize the energy transfer and quenching between adjacent dyes.

Oligo-dye conjugation: There are multiple ways to conjugate multiple different dyes onto the same oligo to achieve different intensity levels: (1) incorporate nucleotide derivatives with different dyes during oligo synthesis; (2) incorporate nucleotides with different epitopes during oligo synthesis and then conjugate the dyes by orthogonal labeling chemistry such as click chemistry; (3) a combination of (1) and (2). In addition, multiple dyes of the same color can be conjugated onto a DNA oligo with preprogrammed sequences using the KREATECH universal linkage system (Leica Biosystems). Thus, to achieve 2-color coding such as 1:2, we can incorporate one red dye at 5′ or 3′ end of each oligo and conjugating 2 green dyes through both internal and end labeling with the KREATECH labeling method. Alternatively, a bright dye like Cy5 (comparing with Cy5.5) and a dimmer dye like Cy3 (comparing with Alexa546) can be paired so that using only 2 dyes per oligo achieves 1:2 intensity ratio. To achieve 3 and 4 color coding such as 1:1:1:1, two kinds of click chemistry ((1) azide and alkyne reaction, (2) tetrazine and vinyl reaction) can be used to label up to 4 different dyes onto the same oligo.

### Example 3

### Screening Intensity Codes with Labeling Scheme-3 and DNA FISH

This example is to use the repetitive DNA sequences in mouse cells to screen good dye pairs for the Labeling Scheme-3. Each dye pair is tested individually in the experiment.

Probe sequences for the repetitive regions of telomere and centromere in mouse genome were ordered from IDT:

Telomere probe, Tel-Cy5-Cy3:
(SEQ ID NO: 1)
CCCTAACCCTAACCCTAA, 5′ labeled with Cy5, 3′
labeled with Cy3;
Centromere probe-1, Cen-Cy5.5-Cy3:
(SEQ ID NO: 1 = 2)
ATTCGTTGGAAACGGGA, 5′ labeled with Cy5.5, 3′
labeled with Cy3;
Centromere probe-2, Cen-Cy5-A532:
(SEQ ID NO: 1 = 3)
ATTCGTTGGAAACGGGA, 5′ labeled with Cy5, 3′
labeled with Alexa532;
Centromere probe-3, Cen-Cy5-A546:
(SEQ ID NO: 1 = 4)
ATTCGTTGGAAACGGGA, 5′ labeled with Cy5, 3′
labeled with Alexa546;
Centromere probe-4, Cen-Cy5-Atto590:
(SEQ ID NO: 1 = 5)
ATTCGTTGGAAACGGGA, 5′ labeled with Cy5, 3′
labeled with Atto590.

DNA FISH on Mouse Embryonic Fibroblast (MEF) cells was performed as follows: MEF cells were seeded on #1.5 glass bottom 8-well chambers and cultured in DMEM supplemented with 10% FBS and 1% penicillin for at least 12 hrs. After removing DMEM solution, MEF cells were immediately fixed with 4% paraformaldehyde (PFA, Electron Microscopy Sciences) for 10 mins. After removing PFA and washing with 2×SSC for 3 times, MEF cells were permeabilized with 70% Ethanol at 4° C. for at least 1 hr. The Ethanol solution was then removed and the MEF cells were washed with 2×SSC for 3 times before submitting to denaturation at 90° C. in 80% formamide (Sigma), 2×SSC for 10 min. After removing denaturation solution, the MEF cells were incubated with 200 uL probe buffer containing 10% formamide, 2× saline-sodium citrate, 20% dextran sulfate (Sigma, MW >500,000) and 200 nM DNA probes at 37° C. for 4 hr. Each well of the chamber was incubated with one kind of probes only to detect intensity ratios of DNA loci. The MEF cells were then washed with 10% formamide in 2×SSC at 37° C. for 2 mins, two times before proceeding to imaging on a spinning disk confocal microscope with laser excitation.

The microscope configuration: The imaging setup including optical filters was the same as that used in Example 1.

Imaging: Multiple dyes with overlapped emission spectra were excited with the same laser and imaged with the same channel at the same time. Basically, all fluorescence excited by laser 640 nm were recorded with the channel of Cy5/700. The fluorescence excited by laser 561 nm were recorded with the channel of Cy3/600.

Image Analysis: The 3D scanned images from the same FOV were first stacked and projected with maximum intensity. This step was done for both the 700 and 600 channels. The max-intensity projected figures were fitted with Gaussian function to obtain its peak intensity and its spatial position in both channels. The fitted spots from 700 channel were then aligned with the spots from 600 channel and deemed as the same telomere or centromere spot if they were separated within three pixels in two channels. The intensities of 700 channel and 600 channel from the same fluorescent spot were then picked to calculate the intensity ratio of these two channels for the spot. Then a 2D (700 and 600 channels) intensity ratio distribution map was created with intensity ratio values of all the fluorescent spots. For those spots appearing in both 700 and 600 channels, we plotted the intensity from 700 channel as x-axis and the intensity from 600 channel as y-axis. In this map, each dot represents an intensity ratio of a FISH spot over two color channels.

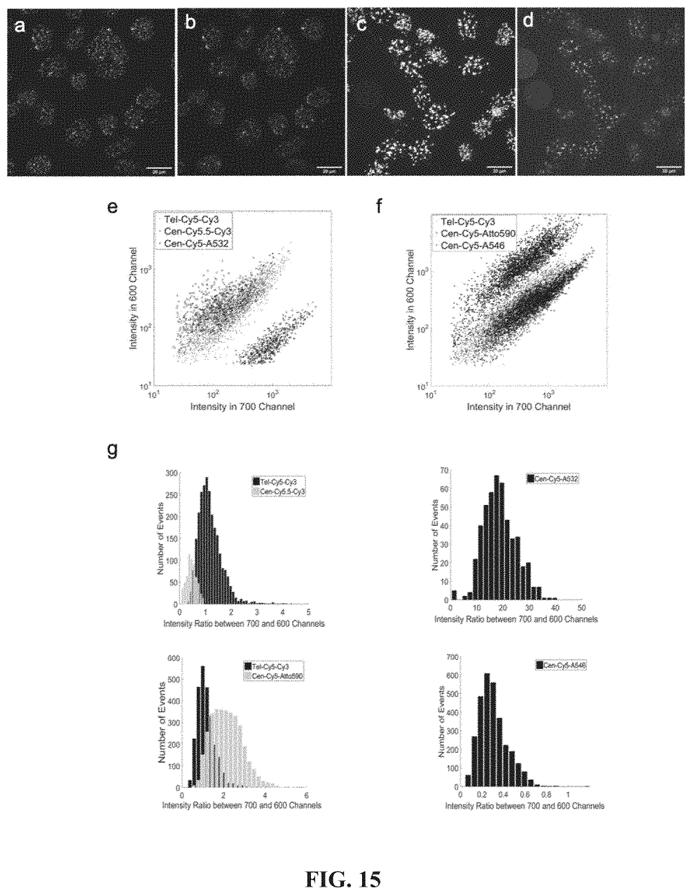

Results and discussion: We calculated the overlapping ratio between dye pairs (listed in the table below) and created a 2D intensity distribution map and 1D intensity ratio histogram (FIG. 15e-g). As shown in FIG. 15e, the 2D intensity distribution of FISH spots labeled by the dye-pair probes of Tel-Cy5-Cy3 and Cen-Cy5-A532 are completely separated from each other. Furthermore, the intensity distribution from the FISH spots of Tel-Cy5-Cy3 is largely distinguishable from that of Cen-Cy5-A546 (FIG. 15f): 9.14% of dots from Tel-Cy5-Cy3 are overlapped with Cen-Cy5-A546 and 22.35% of dots from Cen-Cy5-A546 are overlapped with Tel-Cy5-Cy3 as listed in the table below. Moreover, the intensity distribution of Tel-Cy5-Cy3 is only partially distinguishable from that of Cen-Cy5.5-Cy3: 34.13% of dots from Tel-Cy5-Cy3 are overlapped with Cen-Cy5.5-Cy3 and 82.28% of dots from Cen-Cy5.5-Cy3 are overlapped with Tel-Cy5-Cy3. Intensity distribution of Tel-Cy5-Cy3 is rarely distinguishable from Cen-Cy5-Atto590: 90.86% of dots from Tel-Cy5-Cy3 are overlapped with Cen-Cy5-Atto590 and 85.13% of dots from Cen-Cy5-Atto590 are overlapped with Tel-Cy5-Cy3 (FIG. 15g) as listed in the table below. Lastly, the intensity distributions of Cen-Cy5-A532 and Cen-Cy5.5-Cy3 are completely separated from each other. The intensity distributions of Cen-Cy5-Atto590 and Cen-Cy5-A546 are slightly overlapped with each other (about 2%, FIG. 15g) as listed in the table below.

| | Overlapping ratio of | Overlapping ratio of |
|---|---|---|
| Probes with dye pairs | the first probe | the second probe |
| Tel-Cy5-Cy3\|Cen-Cy5-A532 | 0 | 0 |
| Tel-Cy5-Cy3\|Cen-Cy5-A546 | 9.14% | 22.35% |
| Tel-Cy5-Cy31\|Cen-Cy5.5-Cy3 | 34.13% | 82.28% |
| Tel-Cy5-Cy3\|Cen-Cy5- | 90.86% | 85.13% |
| Atto590 | | |
| Cen-Cy5-A532\|Cen-Cy5.5- | 0 | 0 |
| Cy3 | | |
| Cen-Cy5-Atto590\|Cen-Cy5- | 1.98% | 1.95% |
| A546 | | |

### Example 4

### Multiplex RNA Detection with Labeling Scheme-3 in Cultured Cells

We used the Labeling Scheme-3 to detect 2 RNA targets (PORL2A, CTNNB1) in MEF cells with 2 color channels here. CTNNB1 was labeled with a dye combination of Cy5 and Cy3. POLR2A was labeled with a dye combination of Cy5.5 and Alexa546.

General principle of Probe design: We used indirect labeling together with branched DNA amplification to achieve the Labeling Scheme-3 here. We used 5×5 branched DNA signal amplification for each dye and each primary probe. In this way, each primary probe bound with a primary amplifier with 5 repeats, then each primary amplifier bound with 5 secondary amplifiers where each secondary amplifier had 5 repeats, finally these 5 secondary amplifiers which were associated with the same primary probe bound with up to 25 imaging probes.

Details of 4 tiers of probe design (primary probe, primary amplifier, secondary amplifier, imaging probe): We designed 48 primary probes for each RNA specie. Each primary probe consisted of a 20 nt target-binding sequence which was complementary with the targeted RNA sequence, and a 20 nt readout sequence at both 5′ and 3′ side of the 20 nt target-binding sequence. All primary probes for the same RNA species contained the same readout sequence. Primary probes for different RNA species had different readout sequences and were associated with different primary amplifiers and secondary amplifiers to avoid cross-binding among different kinds of RNA targets. Each primary probe for both two RNA species was bound with a primary amplifier. Each primary amplifier had a 20 nt sequence that was complementary with the readout sequence on the corresponding primary probe. Each primary amplifier had 5 repeats so that it could bind to 5 secondary amplifiers. Each secondary amplifier had a 20 nt sequence that was complementary with the repeat on the corresponding primary amplifier. Each secondary amplifier had 5 repeats so that it could bind to 5 imaging probes. In this way, a 5×5 amplification was achieved for each fluorescent dye. Each imaging probe was 20 nt long with a fluorescent dye on its 5′ end. Two imaging probes were used for CTNNB1: an imaging probe conjugated with one Cy5 dye, another one conjugated with one Cy3 dye. Two imaging probes were used for POLR2A: an imaging probe conjugated with one Cy5.5 dye, another one conjugated with one Alexa546 dye. Each primary probe for CTNNB1 could be associated with up to 25 Cy5 dyes at 5′ end and up to 25 Cy3 dyes at 3′ end. Each primary probe for POLR2A was associated with up to 25 Cy5.5 dyes at 5′ end and up to 25 Alexa546 dyes at 3′ end. Therefore, theoretically, a total of 1200 (48×5×5) Cy5 dye, 1200 Cy3 dye could be associated with an RNA copy of CTNNB1 if the binding efficiency for all probes were 100%. A total of 1200 Cy5.5 dye, 1200 Alexa546 dye were associated with an RNA copy of CTNNB1 if the binding efficiency for all probes were 100%. However, due to insufficient binding, the total number of dye per target might be much lower than this number which increased the intensity variation of the intensity codes.

Design of the target-binding sequences for primary probes: The sequences of RNA targets were extracted from the genome and transcriptome databases such as NCBI or UCSC. Target-binding sequences were required to have GC content within the range of 45-65%. A BLAST query was run on each probe against the whole genome and transcriptome of the specie to get rid of sequences with high redundancy. A minimum probe spacing (>2 nt here) was added and adjusted to facilitate probe binding and minimize fluorophore quenching or FRET. Probe spacing refers to the minimum number of nucleotides between probe binding sites along the RNA target.

The readout sequences of primary probes, the sequences of the primary amplification probes, the sequences of the secondary amplification probes, and the sequences of the imaging probes were designed so that it had minimal non-specific binding to the mouse transcriptome.

Intensity codes: With the oligo probe design described above, we assigned two intensity codes to detect CTNNB1 and POLR2A. As Cy5 was roughly 2 times brighter than Cy5.5 in the 700 channel, Alexa546 was roughly 2 times brighter than Cy3 in the 600 channel in this experiment, the intensity level 1 could be defined as labeling a target with 1200 Cy3 or Cy5.5, and intensity level 2 could be defined as labeling a target with 1200 Alexa 546 or 1200 Cy5. Therefore, CTNNB1 and POLR2A were encoded with 2 intensity codes (1:2) and (2:1), respectively, with the oligo design here. In these two intensity codes, the first digit represented the intensity level in 600 channel, the second digit represented the intensity level in 700 channel.

RNA FISH was performed as follows: MEF cells were seeded on #1.5 glass bottom 8-well chambers (Ibidi) and cultured in DMEM supplemented with 10% FBS and 1% penicillin for at least 12 hrs. After removing DMEM solution, MEF cells were immediately fixed with 4% paraformaldehyde (PFA) for 10 mins. After removing PFA and washing with 2×SSC for 3 times, MEF cells were permeabilized with 70% Ethanol at 4° C. for at least 1 hr. The Ethanol solution was then removed and the MEF cells were washed with 2×SSC for 3 time before submitting to washing buffer (10% formamide and 2×SSC) for 10 mins. After removing washing solution, the MEF cells were incubated with 200 uL probe buffer containing 10% formamide, 2× saline-sodium citrate, 10% dextran sulfate (Sigma, >500,000) and multiple primary DNA oligo pools (IDT, 10 nM for each single primary probe) at 37° C. overnight. Each well of the chamber was incubated with 1-2 kinds of primary probes to detect 1-2 RNA species (PORL2A, CTNNB1) in MEF cells. After overnight incubation, the MEF cells were washed with 10% formamide in 2×SSC at 37° C. for 2 mins, two times. Cells were then incubated with 10 nM primary amplification probes in 200 uL hybridization buffer at 37° C. for 1 hr. After washing in washing buffer for 5 mins, two times, MEF cells were then incubated with 10 nM secondary amplification probes in 200 uL hybridization buffer at 37° C. for 1 hr. After washing in washing buffer for 5 mins, two times, MEF cells were then incubated with 100 nM imaging probes in 200 uL hybridization buffer at 37° C. for 1 hr. After a final round of washing in washing buffer for 5 mins, MEF cells were then incubated with DAPI at a concentration of 0.001 mg/mL for 1 min before proceeding to imaging on a spinning disk confocal microscope with laser excitation (Nikon Ti microscope, Yokogawa CSU-W1 confocal scanner, a sCMOS camera Andor Zyla 4.2, 100× oil objective). Fluorescence imaging was done on the same setup used in Example 1. Each field of view (FOV) was 3D scanned for both 700 channel (640 nm laser excitation with an emission filter of 700/75 m) and 600 channel (561 nm laser excitation with an emission filter of 600/50 m) sequentially. Each fluorescence image was acquired with 200 ms exposure time.

The intensity and position of single FISH spots were extracted in the same way as that used in Example 3 for DNA FISH. For each RNA FISH image, an intensity threshold was carefully selected and optimized to separate non-specific binding signal and background from positive signals. For 2D intensity distribution of RNA spots with 2 dye co-labeling, only spots with intensity above the threshold in both channels were chosen for analysis, otherwise spots were rejected.

Spot Assignment with the Guide of the Reference Intensity Code Map:
1. Generating the reference map: We stained and imaged POLR2A and CTNNB1 separately with their coded intensity ratio. Then we obtained the 2D (700 channel and 600 channel) intensity distribution of all FISH spots of single RNA specie labeling from images of around 100 cells. We could get two separate intensity clusters for POLR2A and CTNNB1, respectively in the same 2D intensity plot.
2. We then tried to find the densest area of each cluster of each dye combination including around 95% of all spots. The algorithm worked in the way that each point was clustered with neighboring point if the distance between the two points was shorter than 25 pixels, which may be user defined and increased to incorporate more sparsely distributed spots. With newly generated cluster of spots representing the densest area of each dye combination, we found the boundary of each cluster by selecting a series of points conforming 2-D boundary that enclosed all the points from each cluster. This boundary represented by a series of points would be used in our next step to define our specific-signal area in the mix-labeled dye combinations.
3. With the boundary for each dye combination determined from the reference map, we selected those spots from mix-labeled clusters that were enclosed by the boundary of the dye combination. By calculating the ratio between the number of spots included in or excluded from the boundary and the total number of spots, we could quantify the specific binding and non-specific binding signal.

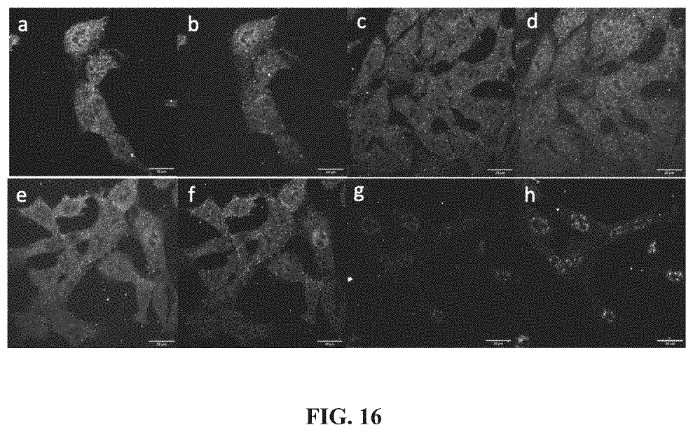

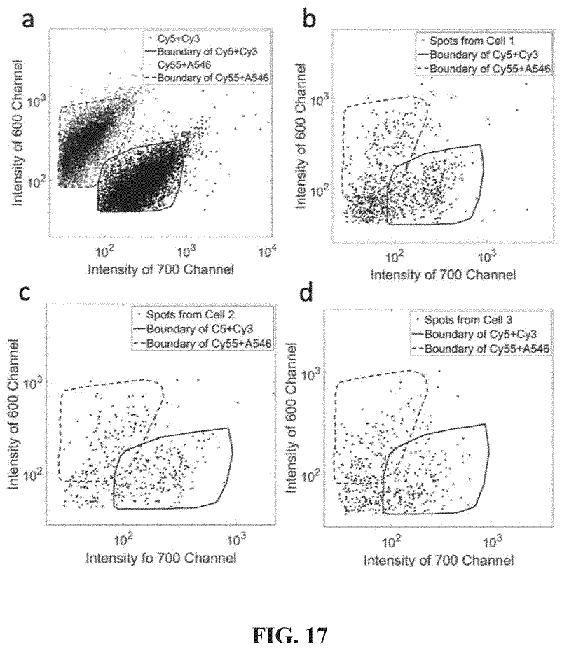

Results and discussion: Here FIG. 16 shows representative raw images of single RNA labeling and two RNA co-labeling. Two intensity codes associated with two dye combinations (Cy5+Cy3, Cy5.5+A546) were successfully separated with each other on the reference map. Therefore, the boundaries of each intensity code were determined successfully. FIG. 17(a) shows the reference map from single RNA staining for two dye combinations with boundaries specified. FIG. 17(b)-(d) shows the application of reference map into the mix-labeled data with three cells shown separately. The table below shows the statistical results for each cell. Spots are either successfully assigned to a RNA target encoded with a unique dye combination (i.e. intensity code) or being dropped due to ambiguity or non-specific binding.

| | | Spots | | Spots of | |
|---|---|---|---|---|---|
| | Spots assigned | assigned to | Spots with | nonspecific | |
| Cells | to Cy55 + A546 | Cy5 + Cy3 | ambiguity | binding | Total spots |
| Cell 1 | 232 (27%) | 362 (42%) | 20 (2%) | 250 (29%) | 864 |
| Cell 2 | 124 (33%) | 185 (49%) | 5 (1%) | 65 (17%) | 379 |
| Cell 3 | 134 (32%) | 171 (41%) | 10 (2%) | 107 (25%) | 422 |

As mRNAs are typically expressed as single copies in mammalian cells, they can be detected by high capacity FISH method developed here. However, due to the limited physical resolution of optical imaging, this method requires each RNA should be separated from each other at a distance of larger than diffraction limited optical resolution (typically around half of the emission wavelength) so that they can be accurately differentiated. If multiple mRNAs are expressed at a high level, they may overlap each other so that the detection accuracy of high capacity FISH imaging is compromised.

### Example 5

### Multiplex RNA Detection with Labeling Scheme-2 in Cultured Cells

We used the Labeling Scheme-2 to detect 2 RNA targets (POLR2A, CTNNB1) in MEF cells with 2 color channels here.

Probe design and synthesis: We used indirect labeling together with branched DNA amplification to achieve the Labeling Scheme-2 here. We designed 48 primary probes for each RNA specie. They were the same as those used in Example 4. We used no (or 1×1) amplification for POLR2A and unbalanced 3×3 amplification for CTNNB1. Each primary probe for POLR2A had a 20 nt readout sequence that hybridized with one imaging probe. Each imaging probe for POLR2A was conjugated with one Cy5 dye at 5′ end and one Cy3 dye at 3′ end. Each primary probe for CTNNB1 had one 20 nt readout sequence at both 5′ and 3′ end so that the 5′ end could hybridize with one primary amplifier and the 3′ end could hybridize with an image probe conjugated with a Cy5 dye directly. The 3′ end of each primary probe for CTNNB1 had no signal amplification. The primary amplifier at 5′ end of each primary probe for CTNNB1 had 3 repeats (20 nt of each repeat) to hybridize with 3 secondary amplifiers. Each secondary amplifier for CTNNB1 had 3 repeats (20 nt of each repeat) to hybridize with 3 imaging probes. Each imaging probe hybridizing with the secondary amplifiers for CTNNB1 was conjugated with a Cy3 dye at 5′ end of the imaging probe. In this way, each primary probe for POLR2A were associated with one Cy5 and one Cy3 dye, indirectly. Each primary probe for CTNNB1 could be associated with one Cy5 and 9 Cy3 dyes, indirectly.

Intensity codes: With the oligo probe design described above, we assigned two intensity codes to detect CTNNB1 and POLR2A. Each copy of CTNNB1 was associated with 48 Cy5 and 432 Cy3 and each copy of POLR2A was associated with 48 Cy5 and 48 Cy3. The intensity level 1 could be defined as labeling a target with 48 Cy3 or Cy5, and the intensity level 2 could be defined as labeling a target with 432 (48*3*3) Cy3 or Cy5. CTNNB1 and POLR2A were encoded with 2 intensity codes (2:1) and (1:1), respectively, with the oligo design here. In these two intensity codes, the first digit represented the intensity level in 600 channel, the second digit represented the intensity level in 700 channel. Intensity code of 2:1 corresponds to an intensity ratio of 9:1. Intensity code of 1:1 corresponds to an intensity ratio of 1:1.

FISH staining, imaging and image analysis were the same as that of Example 4. Using the same FISH spot assignment method as described in Example 4, we could separate the 2D intensity distribution data into two clusters and thus detect 2 RNA species: CTNNB1 and POLR2A.

### Example 6

### Multiplex RNA Detection with Labeling Scheme 2 and 3 in Cultured Cells

This example illustrates how we can combine the Labeling Scheme-2 and Scheme-3 to improve the results of detecting 2 RNA targets (POLR2A, CTNNB1) in Example 4. The same setup including the color channels as Example 4 are used here. CTNNB1 is still labeled with a dye combination of Cy5 and Cy3. POLR2A is still labeled with a dye combination of Cy5.5 and Alexa546. However, we can adjust the number of each dye associated with an RNA target to increase the intensity gap of these two dye combinations so that the intensity codes generated by these two dye combinations can be better separated for higher detection efficiency of both RNA targets.

Strategy-1: Change the number of dye associated with one target. For example, we can still use 5×5 branched DNA amplification for Cy5, Cy3 and Alexa 546, but use only 3×2 branched DNA amplification for Cy5.5 associated with POLR2A so that the number of Cy5.5 dyes with this RNA is decreased by 76% (1-6/25). In this way, when doing co-staining of these two RNA targets, the intensity cluster of POLR2A in the 2D intensity distribution plot (FIG. 17b) will be shifted towards left so that the intensity gap between the cluster for POLR2A and the cluster for CTNNB1 will become larger.

Strategy-2: Change the number of dye associated with both targets simultaneously. For example, we can still use 5×5 branched DNA amplification for Cy5 and Alexa 546, but use only 3×2 branched DNA amplification for Cy5.5 associated with POLR2A and only 3×2 branched DNA amplification for Cy3 associated with CTNNB1 so that the number of Cy5.5 dye for POLR2A and the number of Cy3 dye for CTNNB1 are both decreased by 76% (1-6/25). In this way, when doing co-staining of these two RNA targets, the intensity cluster of POLR2A in the 2D intensity distribution plot (FIG. 17b) will be shifted towards left, and meanwhile the intensity cluster of CTNNB1 in the same plot (FIG. 17b) will be shifted down. In this way, the intensity gap between the cluster for POLR2A and the cluster for CTNNB1 will become even larger than that created by the Strategy-1.

### Example 7

### 4-Plex RNA Detection with Multiple Labeling Schemes in Cultured Cells

We used N=2 and M=2 to achieve 4 plex RNA detection in this example. 3 optical labels were used: Cy5, Cy3, and Alexa 546 dyes. Indirect labeling with branched DNA signal amplification was used here together with multiple labeling schemes to detect 4 RNA species (HOXB1, TFRC, POLR2A, CTNNB1) in MEF cells. As For indirect labeling, primary probes, i.e. the target binding probes, bind to the primary amplification probes first, then to the secondary amplification probes, finally to the imaging probes.

Design of Oligo Probes:

The primary probes could be either 40 nt or 60 nt long, which contained (1) a 20 nt target binding sequence and (2) one or two 20 nt readout sequences at one end or both ends of the target binding sequence as described in Example 4. 48 primary probes were used for each mRNA target. Different combination of dye labeling on imaging probes or different number of imaging probes or the combination of these two was used to achieve different intensity ratios for different targets. The design of primary probes for each target is listed as below:

| mRNA | primary probes |
|---|---|
| HOXB1 | Readout(N20)-target recognition(N20)-Readout(N20), |
| | totally 60 nt |
| POLR2A | Readout(N20)-target recognition(N20)-Readout(N20), |
| | totally 60 nt |
| TFRC | Readout(N20)-target recognition(N20)-Readout(N20), |
| | totally 60 nt |
| CTNNB1 | Readout(N20)-target recognition(N20), totally 40 nt |

N represents oligo sequence.
N20 means 20 nucleotides on the oligo.

In this example, each primary probe for TFRC and HOXB1 has two of the same readout sequences, one at the 5′ end and the other at 3′ end. Each primary probe for POLR2A has two different readout sequences, one at the 5′ end and the other at 3′ end. Each primary probe for CTNNB1 has only one readout sequence at the 5′ end.

In this example, the primary amplifiers for all 4 RNA species are 130 nt long, which contained (1) one 20 nt sequence that bind to the readout sequence of the target binding probe, and (2) five repeats of a 20 nt sequence that binds to the corresponding secondary amplification probe. There is 2 nt space between the repeats. There is also 2 nt space between the first repeat and the sequence that binds to the readout sequence.

In this example, there are two different designs of secondary amplification probes based on that whether the corresponding imaging probes are double-labeled (two dye labeling per imaging probe) or single-labeled (one dye labeling per imaging probe). For secondary amplification probes binding with single-labeled imaging probes, it contains (1) one 20 nt sequence that bind to the corresponding primary amplification probe, and (2) five repeats of a 20 nt sequence that binds to the corresponding imaging probe. There is also 2 nt space between the first repeat and the sequence that binds to the primary amplification probe. This first design is applied to POLR2A, CTNNB1 and TFRC here. For secondary amplification probes binding with double-labeled imaging probes, the corresponding secondary amplification probes contains (1) one 20 nt sequence that bind to the corresponding primary amplification probe, and (2) five repeats of a 20 nt sequence that binds to the corresponding imaging probe, and (3) a 22-nt insert between any two repeats. This second design is applied to HOXB1 here.

In this example, the imaging probe labeled with either (1) one dye on the 5′ or 3′ end (one dye per probe) (2) one dye on both the 5′ and 3′ ends (two dyes per probe). The 4 RNA targets each has its own implementation of indirect labeling in Labeling Scheme 2 and Scheme 3, which corresponds to 4 types of labeling for the imaging probe. The labeling for theses 4 types of imaging probes are listed below:

| | Imaging probes on 5′ side | Imaging probes on 3′ side |
|---|---|---|
| mRNA | of a primary probe | of a primary probe |
| HOXB1 | Cy5-NNNNNNNNNNNNNNNNNNNN- | Cy5-NNNNNNNNNNNNNNNNNNNN- |
| | Cy3 (SEQ ID NO: 6) | Cy3 (SEQ ID NO: 7) |
| Polr2A | Cy5-NNNNNNNNNNNNNNNNNNNN | NNNNNNNNNNNNNNNNNNNN-Alexa |
| | (SEQ ID NO: 8) | 546 (SEQ ID NO: 9) |
| TFRC | Cy3-NNNNNNNNNNNNNNNNNNNN | Cy3-NNNNNNNNNNNNNNNNNNNN |
| | (SEQ ID NO: 10) | (SEQ ID NO: 11) |
| CTNNB1 | Cy5-NNNNNNNNNNNNNNNNNNNN | NA |
| | (SEQ ID NO: 12) | |

N represents oligo sequence

The design of the target recognition sequences for primary probes follows the same principle of oligo design as described in Example 4. The designs of the readout sequences, the sequences of the primary amplification probes, the sequences of the secondary amplification probes, and the sequences of the imaging probes follow the same principle of oligo design as described in Example 4 as well.

In this example, we use four intensity codes to detect four RNA targets: 2:2, 0:1, 2:1, and 2:0. In this example each mRNA target binds to 48 target binding probes. Therefore, in this example, the signal of each target binding probe is amplified either by 5*5=25 or by 5*5*2=50. The amplification of each set of target binding probe is listed as below:

| | Amplified through | Amplified through | |
|---|---|---|---|
| | 5′ readout sequence | 3′ readout sequence | Amplification |
| mRNA | for each dye | for each dye | for each dye |
| HOXB1 | 5*5 = 25 | 5*5 = 25 | 25 + 25 = 50 |
| Polr2A | 5*5 = 25 | 5*5 = 25 | 25 |
| TFRC | 5*5 = 25 | 5*5 = 25 | 25 + 25 = 50 |
| CTNNB1 | 5*5 = 25 | 0 | 25 |

The intensity level 1 is defined as labeling a target with 1200 Cy3 or Cy5 (25*48=1200), and intensity level 2 is defined as labeling a target with 1200 Alexa 546 or 2400 Cy3 for the Cy3/600 channel, or 2400 Cy5 for the Cy5/700 channel.

The intensity ratio code for each mRNA is listed as below. 2 intensity levels (1, 2) are used in the 600 fluorescence channel for Cy3 and Alexa 546, and 2 intensity levels (1 and 2) are used in the 700 fluorescence channel for Cy5.

| | | | | | # of | |
|---|---|---|---|---|---|---|
| | | | # of | # of | Alexa | Intensity |
| | | Length | Cy3 | Cy5 | 546 | Ratio |
| | | of | dyes | dyes | dyes | (600 |
| | NCBI | mRNA | per | per | per | channel:700 |
| mRNA | Accession # | (nt) | target | target | target | channel) |
| HOXB1 | NM_008266 | 2077 | 2400 | 2400 | 0 | 2:2 (1:1) |
| Polr2A | NM_001291068 | 6736 | 0 | 1200 | 1200 | 2:1 |
| TFRC | NM_011638 | 4920 | 2400 | 0 | 0 | 2:0 |
| CTNNB1 | NM_007614 | 3640 | 0 | 1200 | 0 | 0:1 |

Synthesis of oligo probes: All oligo probes were ordered from IDT.

FISH staining and imaging process were conducted in same way as described in Example 4. For imaging analysis, 2D intensity distribution of all FISH spots were obtained with the same approach used in Example 3 for DNA FISH. After that, these spots were divided into two clusters with the Expectation-Maximum algorithm without using a reference intensity code map. Two clusters coded with 2 digit intensity ratios were successfully separated. Each of these two clusters represents one RNA specie. We assigned the cluster above to POLR2A and the other below to HOXB1 according to the intensity distribution of positive control with single RNA labeling.

Results and Discussion:

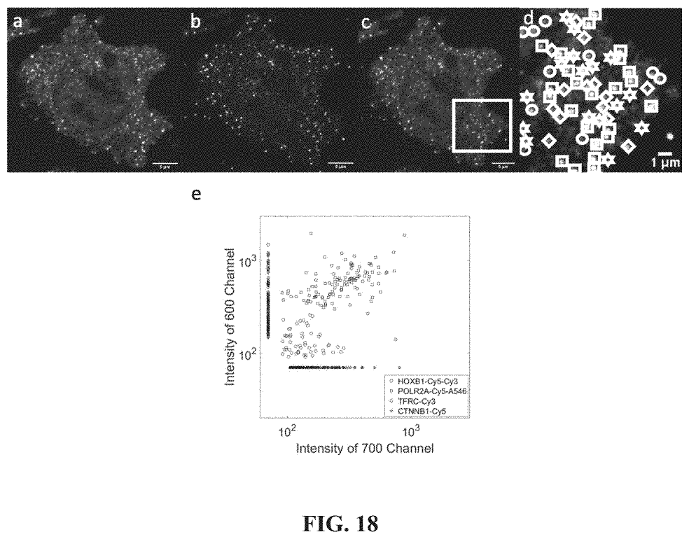

The resulting confocal images of 700 and 600 channels of multiplexed labeling of MEF cell are shown in FIG. 18a and FIG. 18b, respectively. The overlapping image and one zoomed-in area are shown in FIG. 18c and FIG. 18d. Different shapes are shown in FIG. 18d to mark the position of each RNA, where HOXB1 is marked with circle, POLR2A is marked with square, TFRC is marked with diamond and CTNNB1 is marked with hexagram. In this cell, 54 HOXB1 spots, 117 POLR2A spots, 157 CTNNB1 spots and 136 TFRC spots are detected, which are close to copy number counts determined by conventional single molecule RNA FISH without intensity coding.

The corresponding 2D intensity map in log scale is shown in FIG. 18e where 4 clusters of dots are clearly distinguished from each other. Using the probe design for HOXB1 in this example, the designed intensity levels for HOXB1 and POLR2A in Cy3 channel were the same and their intensity ratio distribution might not be separated from each other. However, according to the 2D intensity distribution plot in FIG. 18e, two clusters are successfully separated. By comparing with the positive control results, the intensity of HOXB1 is lower than that of the intensity of POLR2A in Cy3/600 channel. We assigned the cluster above to POLR2A and the other below to HOXB1. We conclude that dye quenching may exist for the imaging probe with the dye pair labeling scheme of Cy3-Cy5 here, which is the application of the Labeling Scheme-4. This is further confirmed by the positive control result of staining HOXB1 alone with the same dye combination in the same cell line. Therefore, the designed intensity code for HOXB1 is actually 1:1 instead of 2:2. In other words, the intensity code of 2:2 and 1:1 in this case overlap with each other and cannot be separated well.

Since CTNNB1 are only labeled with cy5 and only shown in 700 channel, they stand out from HOXB1 and POLR2A which are shown in both 700 and 600 channels. Similar rule applies to TFRC which are labeled with cy3 and only shown in 600 channel.

To summarize, four unique clusters representing four different targets are successfully identified using a combination of Labeling Scheme-2, Scheme-3 and Scheme-4.

### Example 8

### Multiplex RNA Detection with Labeling Scheme-3 and Differentiated Labeling

We combined the Labeling Scheme-3 and differentiated labeling here to detect 2 RNA targets (POLR2A, MTOR) in MEF cells with 2 color channels here.

We designed 60 primary probes to label POLR2A, 60 primary probes for MTOR. Each primary probe was 40 nt long, 20 nt of which was a target-binding sequence. Each target-binding sequence of the primary probe was connected with a 20 nt readout sequence at its 5′ end. Similar to Example-4, we used 5×5 branched DNA amplification for each primary probe. Therefore, each primary probes was bound to a primary amplifier with 5 repeats first, then to 25 secondary amplifiers, finally to 25 imaging probes labeled with a dye.

Differentiated labeling: To implement Labeling Scheme-3, Cy5 and Cy3 are combined to label POLR2A, Cy5.5 and Alexa 546 are combined to label MTOR. In this way, POLR2A and MTOR are labeled with 2 different intensity ratios. The primary probes for POLR2A were separated into two groups: 30 probes were labeled indirectly with Cy5 dyes at 5′ end, 30 probes with Cy3 dyes at 5′ end. The primary probes for MTOR were separated into two groups: 30 probes were labeled indirectly with Cy5.5 dyes at 5′ end, 30 probes with Alexa546 dyes at 5′ end. Therefore, we used partially overlapped dyes Cy5 and Cy5.5 in 700 channel and partially overlapped dyes Cy3 and Alexa546 in 600 channels to detect these two RNA species, which was the application of the Labeling Scheme-3. Each copy of MTOR RNA was designed to be associated with up to 750 (30*5*5) Cy5.5 dyes and 750 Alexa546 dyes. Each copy of POLR2A was designed to be associated with up to 750 (30*5*5) Cy5 dyes and 750 Cy3 dyes.

FISH staining, imaging and image analysis were the same as that of Example 4. With the design in this example, POLR2A should separate well from MTOR on the 2D intensity distribution plot.

### Example 9

### Multiplex RNA Detection with Labeling Scheme-1 and Scheme-3

We combined the Labeling Scheme-1 and Scheme-3 here to detect 2 RNA targets (MTOR, CTNNB1) in MEF cells with 2 color channels here. We used different number of primary probes to label these two RNA species, which was the application of the Labeling Scheme-1. We used partially overlapped dyes Cy5 and Cy5.5 in 700 channel and partially overlapped dyes Cy3 and Alexa546 in 600 channels to detect these two RNA species, which was the application of the Labeling Scheme-3.

Probe design and synthesis: We used an indirect labeling together with branched DNA amplification here. Different from Example-4, we designed only 24 primary probes for CTNNB1 and 96 probes for MTOR. Other than the number of primary probes for RNA targets were different, all other probe design for both RNAs was the same as that in Example-4. In other words, MTOR was labeled by Cy5 and Cy3. CTNNB1 was still labeled by Cy5.5 and Alexa546. Each primary probe was amplified 5×5 to be associated with 25 Cy5 dyes at 5′ end and 25 Cy3 dyes at 3′ end. If all probe hybridization efficiency was 100%, each copy of MTOR could be associated with up to 2400 (96*5*5) Cy5 and 2400 Cy3 dye, and each copy of CTNNB1 could be associated with up to 600 (24*5*5) Cy5.5 and 600 Alexa546 dye.

FISH staining, imaging and image analysis were the same as that of Example 4. We expected that POLR2A should separate very well from CTNNB1 on the 2D intensity distribution plot. Comparing with the 2D intensity distribution of POLR2A in Example 4, MTOR here should shift towards upper right due to higher number of primary probes. Comparing with the 2D intensity distribution of CTNNB1 in Example 4, CTNNB1 here should shift towards lower left due to lower number of primary probes.

### Example 10

### Multiplex RNA Detection with Alternative Labeling Scheme-3

We demonstrate here how to just use one dye per target approach to achieve the Alternative Labeling Scheme-3.

We can use Alexa 647 to label POLR2A mRNA and Alexa 700 to label CTNNB1 mRNA, respectively. These two dyes have overlapped emission spectra as illustrated in FIG. 10. Each copy of POLR2A is labeled with 48 primary probes together with 5×5 branched DNA amplification. Each copy of CTNNB1 is labeled with 48 primary probes together with 5×5 branched DNA amplification. The imaging probe for POLR2A is labeled with one Alexa 647 dye. The imaging probe for CTNNB1 is labeled with one Alexa 700 dye. All the sequence design for each tier of probes (including primary probes, primary amplifiers, secondary amplifiers, imaging probes) is the same as that used in Example 4.

We customize two emission filters to record the fluorescence of Alexa 647 and Alexa 700: 650-710 nm, 745-805 nm. Both dyes are excited by the same laser line of 633 nm. In the color channel of 650-710 nm, Alexa 647 emits significant higher intensity than Alexa 700 when using the same number of dyes per target for both RNA species. In the color channels of 745-805 nm, Alexa 700 can emit higher intensity than that of Alexa 647. When doing imaging, use the same excitation of 633 nm and detect the fluorescence in two color channels with two emission filters described above sequentially or simultaneously, two color images are recorded. In this way, Alexa 647 and Alexa 700 can generate two different intensity ratios over the same two color channels so that POLR2A and CTNNB1 can be distinguished. By calculating the spectrum area in a color channel for each dye, we estimate the intensity ratio of Alexa 647 in the channel of 650-710 nm and 745-805 nm is 3:1 (the first digit is the intensity in the channel of 650-710 nm), and the intensity ratio of Alexa 700 is 1:2.85 (the first digit is the intensity in the channel of 650-710 nm). Therefore, POLR2A and CTNNB1 can be differentiated by these two dyes together with two channels designed above. To further adjust the intensity distribution of each target so that the intensity clusters of these two targets can be better separated, we can adjust the number of Alexa 647 associated with POLR2A, or the number of Alexa 700 associated with CTNNB1, or both numbers. Moreover, we can change the filter design (e.g. increasing or shortening the emission window of an emission filter, or changing the photon transmission efficiency of an emission filter) to adjust the intensity ratio of each dye across two color channels for better RNA detection efficiency.

Other than the dye labeling and optical filters described above, all other processes including probe design, FISH staining experiment, imaging and image analysis can be done in the same approach in Example 4.

### Example 11

### Multiplex RNA Detection with 4 Color Channels

In this example, we plan to use 3 color channels (N=3, M=2) for intensity coding, another 1 color channel for error correction of non-specific binding. The 3 color channels for intensity coding are the same used in Example-1. Other than that, we add one more color channel to detect Cy7 dye. 12 mouse mRNA genes are used for test here: TFRC, MTOR, EGFR, HER2, TBP, TOP1, PRDM4, BRAC1, POLR2A, STAT3, NOTCH1, WNT11.

Probe design and dye labeling: Mouse RNA sequences can be obtained from the NCBI gene database. For each RNA, depending on its length, we design 24-48 primary probes for intensity coding. We design additional 24-48 primary probes for each RNA and error-correction. 6 dyes are used here for intensity coding: Cy5, Cy5.5, Cy3, Alexa546, Alexa488 and Alexa514. Cy7 dye is used for error correction of non-specific binding only but not for intensity coding. Branched DNA amplification is also combined to finely adjust the intensity level and variation of each intensity code. Therefore, a combination of Labeling Scheme-1, Scheme-2, Scheme-3 and alternative schemes for error correction are integrated to achieve the best performance of RNA detection efficiency.

Intensity Coding: We use 2 intensity levels and 3 colors to detect 12 RNA species. 12 intensity codes are assigned to 12 RNA species in a way that codes with more digits are assigned to longer RNAs as listed in the table below. For example, 3 digit code 1:1:2 is assigned to label longer RNA POLR2A, 1 digit code 1:0:0 is assigned to label shorter RNA AKT1. For each intensity code in this table, the first digit is Cy5/700 channel, the second digit is Cy3/600 channel, the third digit is Alexa488/500 channel. E.g. intensity code 1:0:0 represented the first color is Cy5 with intensity level 1, while the second and third color have no intensity; 1:2:1 represents the first color is Cy5 with intensity level 1, the second color was Cy3 with intensity level 2, the third color was Atto488 with intensity level 1.

| mRNA Name | Length | CDS | Intensity Code |
|---|---|---|---|
| AKT1 | 2707 | 371-1813 | 1:0:0 |
| WNT11 | 3022 | 498-1562 | 0:1:0 |
| PRDM4 | 3797 | 208-2619 | 0:0:1 |
| TOP1 | 3859 | 334-2637 | 0:1:2 |
| Stat3 | 4520 | 309-2621 | 0:2:1 |
| TFRC | 4920 | 165-2456 | 1:2:0 |
| HER2 | 4998 | 180-3950 | 2:1:0 |
| BRCA1 | 5549 | 111-5549 | 2:0:1 |
| EGFR | 5983 | 281-3913 | 1:0:2 |
| Polr2A | 6736 | 411-6323 | 1:1:2 |
| MTOR | 8612 | 121-7770 | 1:2:1 |
| NOTCH1 | 9497 | 265-7860 | 2:1:1 |

Setup and FISH Experiment: We can use a spinning disk confocal microscope (Nikon, Ti-E) and Andor sCMOS camera to image MEF cells stained with probes. The microscope is installed with 5 lasers and 5 color channels: 405 nm laser with an emission filter at 450/50 m; 488 nm laser with an emission filter at 525/50 m, 561 nm laser with an emission filter at 600/50 m; 640 nm laser with an emission filter at 700/75 m; 750 nm laser with an emission filer at 810/90 m. FISH staining experiment can be done in the same way used in Example 4.

Image Analysis: Single molecule intensity distribution can be analyzed in the same way used in Example 4.

### Example 12

### Multiplex DNA Detection with Labeling Scheme 1 in Cultured Cells

To implement Scheme I, we designed two sets of primary probes, which are constructed from the following regions, Chr21: 18627683-18727683 (100 kb in total, denoted as SI1) and Chr19: 29120001-29220001 (100-kb in total, denoted as SI2). The 42-nt target sequences complementary to the above genomic region of interest were selected to cover SI1 with 1000 probes tiling over the 100-kb genomic region and SI2 with 1000 probes tiling over the 100-kb genomic region. We then flank each primary probe on the 3′ side with a readout sequence for branched DNA signal amplification. We used 3×3 branched DNA amplification for all primary probes of both targets. Amplification probes was designed in the same way as that of Example 4 for RNA FISH except each primary and secondary amplifier contained only 3 repeats for amplification. In addition, for the 1000 probes targeting SI1, 100 of them were labeled with Cy5 dyes indirectly by signal amplification, and 900 of them are labeled with Cy3 dyes indirectly by signal amplification. For the 1000 probes targeting SI2, 100 of them were labeled with Cy3 dyes indirectly by signal amplification, and 900 of them are labeled with Cy5 dyes indirectly by signal amplification.

Intensity coding: Here, we define the first intensity level as 900 (100*3*3) Cy3 or Cy5 dyes, the second intensity level as 8100 (900*3*3) Cy3 or Cy5 dyes. Based on the oligo design above, SI1 is coded with an intensity code of 2:1 (600 channel:700 channel), corresponding with an designed dye ratio of 9:1 (Cy3:Cy5). SI2 is coded with an intensity code of 1:2 (600 channel:700 channel), corresponding with an designed dye ratio of 1:9 (Cy3:Cy5).

FISH experiment, imaging and image analysis: We used HeLa cells for DNA FISH here. The staining protocol was the same as that of Example 3 except more tiers of intermediary probes were added after primary probes bound to the targets. The hybridization and washing condition for intermediary probe binding was the same as imaging probe binding in Example 3. Imaging process and image analysis could be done in the same way as that of Example 3.

### Example 13

### Multiplex DNA Detection with Labeling Scheme 1 and 3 in Primary Tissue

We designed probes to detect two chromatin loci (telomere and centromere) in mouse brain tissue and human PBMC cells here. We designed a probe to recognize the repetitive sequences of telomere and another probe to recognize the repetitive sequences of centromere. As different telomere or centromere loci on different mouse or human chromosomes had various number of repeat units, they could bind with different number of telomere or centromere probes, which was the application of the Labeling Scheme-1. Meanwhile, partially overlapped dyes with different fluorescence intensity were used here to differentiate these two DNA segments, which was the application of the Labeling Scheme-3.

For mouse tissue sample, the brain tissue from C57BL/6 mouse was used here. Typically, the telomere length in this mouse strain varies between 10 kb-50 kb. So in principle, hundreds to thousands of telomere repeat units may exist in each telomere locus. Mouse centromeres of different chromosomes vary between 100 kb-1Mb.

For human cells, the telomere length varies between 1 kb -10 kb. Human centromeres among different chromosomes vary between 0.5-10 Mbp.

Probes for Telomere and Centromere Labeling in Mouse Tissue:
Probe sequences for the repetitive regions of Telomere and Centromere in mouse genome were ordered from IDT:

Telomere probe, Tel-Cy5-Cy3:
(SEQ ID NO: 1)
CCCTAACCCTAACCCTAA, 5′ labeled with Cy5, 3′
labeled with Cy3
Centromere probe, Cen-Cy5-A532:
(SEQ ID NO: 2)
ATTCGTTGGAAACGGGA, 5′ labeled with Cy5, 3′
labeled with Alexa532.

Probes for a repetitive sequence of chromosome-1 and the centromere in human cells: Chromosome-1 probe Chr1-TYE665-Cy3 for a repetitive DNA segment on chromosome 1 (Ch1-Re): CCAGGTGAGCATCTGACAGCC (SEQ ID NO:13), 5′ labeled with TYE665, 3′ labeled with Cy3; Centromere probe, Cen-Cy5-A546: ATTCGTTGGAAACGGGA (SEQ ID NO:2), 5′ labeled with Cy5, 3′ labeled with Alexa546. The concentration of each probe in the hybridization buffer was 200 nM.

A DNA FISH on mouse brain tissue was performed as follows: A block of mouse brain tissue (ordered from Cell Biologics) was embedded with OCT and cryo-cut into 8 um sections. Sections were attached onto poly-lysine (PLL, CultreX) coated grid coverslips (Gridded glass coverslips Grid-500, #1.5H (170 um) D 263 Schott glass, ibidi). The tissue section was immediately fixed with 4% PFA for 12 mins at RT. The fixed tissue was then washed with 2×SSC for 3 times and permeabilized with 70% Ethanol at RT for at least 1 hr. The Ethanol solution was then removed and the tissue was washed with 2×SSC for 3 time before submitting to denaturation at 90° C. in 80% formamide, 2×SSC for 10 min. After denaturation, the tissue was incubated with 200 uL probe buffer containing 10% formamide, 2× saline-sodium citrate, 20% dextran sulfate (Sigma, >500,000) and 200 nM DNA probes at 37° C. for 4 hr. Both telomere and centromere probes were used at a concentration of 200 nM here. The tissue was then washed with 10% formamide in 2×SSC at 37° C. for 2 mins, two times before imaged on the same setup used in Example-16. Each FOV was 3D scanned to cover all the signals and each fluorescence image was acquired with 200 ms exposure time. Cy5 dyes were imaged with the color channel of 700 with an emission filter of 700/75 m, Cy3 and Alexa532 dyes were imaged with the color channel of 600 with an emission filter of 600/50 m.

A DNA FISH on human PBMC cells was performed as follows: Human PBMC cells isolated from whole blood fixed in a combination of 3:1 (v/v) methanol and acetic acid solution. After that, cells were attached onto #1.5 glass bottom 8-well chambers and washed with 2×SSC for 3 times before submitting to denaturation at 90° C. in 80% formamide, 2×SSC for 10 min. With denaturation solution removed, the blood cells were incubated with 200 uL probe buffer containing 10% formamide, 2×SSC, 20% dextran sulfate and 200 nM DNA probes at 37° C. for 1 hr. The blood cells were then washed with 10% formamide in 2×SSC at 37° C. for 2 mins, two times before imaging on a spinning disk confocal microscope, which is the same as that in Example-16. Each FOV was 3D scanned to cover all the signals and each fluorescence image was acquired with 200 ms exposure time.

Image Analysis: The imaging data from mouse and human blood cells were analyzed in the same approach. The 3D scanned images from the same FOV were first stacked and projected with maximum intensity. This step was done for both the 700 and 600 channels. The max-intensity projected figures were fitted with Gaussian function to obtain its peak intensity and its 2D position for both the 700 and 600 channels. The fitted dots from 700 channel were then aligned with the dots from 600 channel and deemed as from the same Telomere or Centromere spot if they were located within three pixels. The intensities of 700 and 600 channels from the same spot were then picked for downstream analysis. A separation line was then drawn according to the apparent separation between two clusters of dots. Each spot was then assigned to either telomere or centromere based on their sole intensity ratio distributions. Telomere and centromere in the same image were then marked differently based on the assignment above.

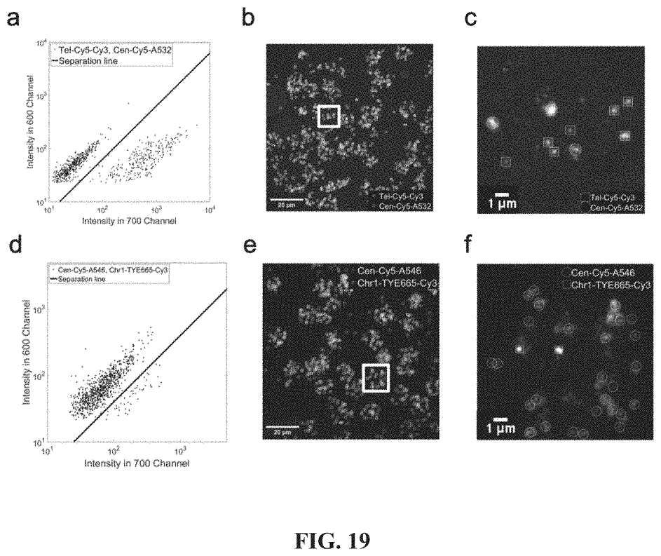

Results and discussion for DNA FISH in mouse tissue: Based on the characteristics of the intensity distribution of each dye pair obtained in Example 3, we chose to combine two dye pairs Cy5-Cy3 and Cy5-A532 probes to detect telomere and centromere simultaneously. The clusters of intensity dots from each dye pair were separated from each other as expected (FIG. 19a). The identification of each telomere and centromere spots in a DNA FISH image was shown in FIG. 19b-c.

Results and discussion for DNA FISH in human cells: Based on the characteristics of the intensity distribution of each dye pair obtained in Example 3, we chose to combine two dye pairs Cy5-Alexa546 and TYE665-Cy3 to label two DNA repetitive sequences Ch1-Re and centromere simultaneously. The clusters of intensity dots from each dye pair were separated from each other as expected (FIG. 19d). The identification of Ch1-Re and centromere spots in one DNA FISH image was shown in FIG. 19e-f. In FIG. 19f, two spots from Ch1-Re were successfully separated from all the other centromere spots by intensity ratio coding.

### Example 14

### DNA FISH with Labeling Scheme-2 and Scheme-4 in Cultured Cells

In this example, we demonstrate how the Labeling Scheme-2 and Scheme-4 can be used to adjust the intensity level of the same target.

Probes: We designed 3 sets of oligo probes to achieve 3 different intensity ratios for the same telomere target: 1 Cy5:2 Cy3, 1 Cy5:10 Cy3, 1 Cy5:30 Cy3. The probe sequence for telomere was: CCCTAACCCTAACCCTAA. Probe set-1 included a primary probe, an imaging probe. Probe set-2 included a primary probe, a primary amplifier, an imaging probe. Probe set-3 included a primary probe, a primary amplifier, an imaging probe. All three primary probes shared the same target-binding sequence for the repetitive regions of telomere in human genome, which was the same as that used in Example 13. All three primary probes were labeled with one Cy5 dye internally (3′ end of the target-binding sequence). All primary probes had two different 20 nt readout sequences, one at 5′ and the other at 3′ end. Each primary probe for the probe set-1 bound with two imaging probes of 20 nt, one at 5′ and the other at 3′ end. Each imaging probe for the probe set-1 had 1 Cy3 label at the 3′ end of the probe. The primary probes for the probe set-2 and set-3 had the same sequence as the primary probe for the probe set-1. Each primary probe for the probe set-2 and set-3 bound to 2 different primary amplifiers, one at 5′ and the other at 3′ end. Each primary amplifier for the probe set-2 had a 20 nt sequence to be complementary with the readout sequence of the primary probe for this set and 5 repeats of 20 nt to bind with 5 imaging probes. Each imaging probe for the probe set-2 had a 20 nt sequence and was labeled with a Cy3 dye at 3′ end. Each primary amplifier for the probe set-3 had a 20 nt sequence to be complementary with the readout sequence of the primary probe for this set and 15 repeats of 20 nt to bind with 15 imaging probes. Each imaging probe for the probe set-3 had a 20 nt sequence and was labeled with a Cy3 dye at 3′ end. In this way, each primary probe for the probe set-1 can be associated with 1 Cy5 and 2 Cy3. Each primary probe for the probe set-2 can be associated with 1 Cy5 and 10 Cy3. Each primary probe for the probe set-3 can be associated with 1 Cy5 and 30 Cy3.

DNA FISH experiment: HeLa cells were attached onto a poly-lysine (PLL, CultreX) coated glass-bottom 8 well chamber (#1.5H (170 um) D 263 Schott glass, ibidi). 3 batches of cells were attached into 3 different wells to be stained with 3 different sets of probes. In each well, FISH staining experiment was done in the following order: Cells were immediately fixed with 4% PFA for 5-10 mins at RT. The fixed cells were then washed with 2×SSC 3 times and permeabilized with 70% Ethanol at RT for at least 1 hr. The Ethanol solution was then removed and cells were washed with 2×SSC for 3 time before submitting to denaturation at 90° C. in 80% formamide, 2×SSC for 10 min. After denaturation, cells were incubated with 200 uL probe buffer containing 10% formamide, 2× saline-sodium citrate, 20% dextran sulfate (Sigma, >500,000) and 200 nM primary probes at 37° C. for at least 4 hr. HeLa cells were washed with 10% formamide in 2×SSC at 37° C. for 2 mins, two times. Cells were then incubated with 10 nM primary amplification probes in 200 uL hybridization buffer at 37° C. for 1 hr (If no primary amplifiers were used, this step was skipped). After washing in washing buffer for 5 mins, two times, HeLa cells were then incubated with 100 nM imaging probes in 200 uL hybridization buffer at 37° C. for 1 hr. After a final round of washing in washing buffer for 5 mins, cells were then incubated with DAPI at a concentration of 0.001 mg/mL for 1 min before proceeding to imaging on a spinning disk confocal microscope with laser excitation (Nikon Ti microscope, Yokogawa CSU-W1 confocal scanner, a sCMOS camera Andor Zyla 4.2, 100× oil objective). Fluorescence imaging was done on the same setup used in Example 1. Each field of view (FOV) was 3D scanned for both 700 channel (640 nm laser excitation with an emission filter of 700/75 m) and 600 channel (561 nm laser excitation with an emission filter of 600/50 m) sequentially. Each fluorescence image was acquired with 200 ms exposure time.

Image analysis was done in the same approach used in Example-3 for DNA FISH.

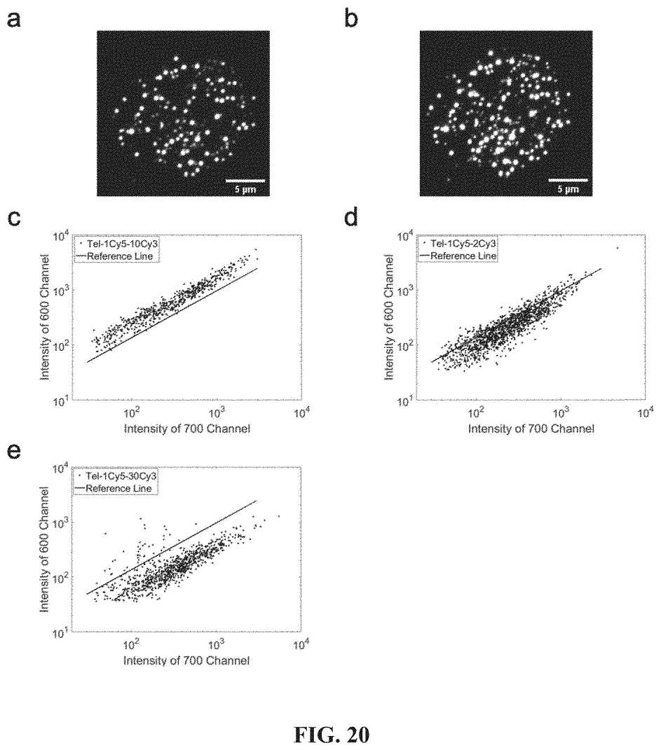

Results and Discussion: FIG. 20c-e shows the results of the intensity distribution of three programmed intensity ratios. They are all compared with the same reference line. Comparing FIG. 20c with FIG. 20d, the intensity of telomere staining with a ratio of 1Cy5:10Cy3 in Cy3/600 channel is mostly above the reference line and higher than 1Cy5:2Cy3, indicating that increasing the number of Cy3 dye per target can increase the intensity in Cy3/600 channel. In FIG. 20e, the intensity of telomere staining with a ratio of 1Cy5:30Cy3 is mostly below the reference line and much lower than 1Cy5:10Cy3 in FIG. 20c, indicating that increasing the number of dye per target too much may decrease the intensity in Cy3/600 channel due to the crowded quenching effect as dyes quench each other when they come together very closely.

### Example 15

### Multiplex Protein Detection for Cell Type Analysis

This example demonstrates how to use intensity coding to barcode multiple protein biomarkers to detect different cell types (FIG. 13a). Each protein biomarker represents one cell type in this example. Protein targets used in this example were CD34, CD45, CD9, Pancytokeratin. Each protein marker represents a distinct cell type.

For multiplex detection of proteins, we can use (1) N=2 and M=2 (Labeling Scheme-2) with Cy5 and Cy3 dyes and (2) N=2 and M=2 (Labeling Scheme-3) with Cy5, Cy3, Alexa546, and Cy5.5 dyes to achieve 4 plex protein detection, respectively. Indirect labeling and signal amplification are used for each labeling scheme. For such indirect labeling as shown in FIG. 12b-c, the primary probes (i.e. target binding probes), which link to the targets directly, bind to secondary probes first, then are associated with the primary amplification probes and the secondary amplification probes. The dye-labeled imaging probes bind to the corresponding secondary amplification probes.

Design of antibody-oligo conjugate: As shown in FIG. 12, an antibody-oligo conjugate is composed of an antibody, an oligo which works as a primary probe, and a linker that connects the oligo to the antibody to form the conjugate.

Antibodies for antibody-oligo conjugate: Primary antibodies against target proteins and used for making the antibody-oligo conjugate were ordered from vendors. The antibodies should be purified before conjugation.

Design of linkers for antibody-oligo conjugate: A linker can be a compound or polymer that is added to an antibody when activating the antibody for conjugation. The linker can also be a compound or polymer that is added to the oligo when activating the oligo for conjugation. The linker can also be part of the oligo when the oligo is synthesized. The length of the linker can be flexible. In this example, the linker is a poly T (10 T's), which will be synthesized as part of the oligo to be conjugated to an antibody.

Design of barcoding oligos for antibody-oligo conjugate: For specificity, the oligos used for antibody-oligo conjugation contain sequence that is not found in the organism of study. Each type of antibodies is conjugated to oligos with sequences that is unique to that type of antibodies and function as an intensity barcode for the target of the antibody. The oligo contains a poly T sequence (10 T's) as a linker between the antibody and the oligo that is unique to each kind of target. For conjugation, the oligo has an amine group on the 5′ end.

Design of oligo probes for intensity coding and signal amplification:

The oligo sequences of primary probes, secondary probes, primary amplification probes, secondary amplification probes, and imaging probes for the indirect labeling (with amplification) in Labeling Schemes 2, and 3 are designed in a similar way as that of Example 4 so that they have minimal non-specific binding to the RNA transcriptome and genome of the targeted species.

In the indirect labeling (with amplification) of Labeling Schemes of 2, and 3, all the secondary probes contain (1) one 20 nt primary probe binding sequence that bind to a unique region of the barcoded primary probe or target-binding oligo, and (2) one 20 nt readout sequence on one end of the primary probe binding sequence for one digit intensity code or one 20 nt readout sequences on each end of the primary probe binding sequence for two digits intensity codes. Although each primary probe in FIGS. 12b and 12c bind to only one secondary probe for all the targets, but in this example, each primary probe bind to 6 secondary probes for all four targets. Imaging probes (all 20 nt) bound to the secondary amplification probe to indirectly label the target. Each imaging probe is labeled with one dye only. For CD9 and CD34 with 2 digits intensity codes in Labeling Scheme-2, unbalanced amplification is used to amplify the dyes in Cy5/700 and Cy3/600 channels. The amplification number of probes and dye choice for each target and labeling scheme are listed in the tables below.

| | | | Cy5 | | Cy3 | | |
|---|---|---|---|---|---|---|---|
| | | | # of | # of | # of | # of | Intensity |
| | | | repeats on | repeats on | repeats on | repeats on | Code |
| Labeling | | Dye | primary | secondary | primary | secondary | (Cy3/600: |
| Scheme | Target | choice | amplifier | amplifier | amplifier | amplifier | Cy5/700) |
| 2 | CD9 | Cy5, | 4 | 4 | 2 | 1 | 1:2 |
| | | Cy3 | | | | | |
| | CD34 | Cy5, | 2 | 1 | 4 | 4 | 2:1 |
| | | Cy3 | | | | | |
| | CD45 | Cy5 | 4 | 4 | 0 | 0 | 0:2 |
| | Pancyto | Cy3 | 0 | 0 | 4 | 4 | 2:0 |
| | keratin | | | | | | |

With the probe design and intensity codes used in the tables above, intensity level 1 using the Labeling Scheme-2 was defined as 12 (6*2*1) Cy3 or Cy5 dyes in Cy3 or Cy5 channel. Intensity level 2 using the Labeling Scheme-2 was defined as 96 (6*4*4) Cy3 or Cy5 dyes in Cy3 or Cy5 channel.

For CD9 and CD34 with 2 digits intensity codes in Labeling Scheme-3, balanced amplification is used to amplify the dyes in Cy5/700 and Cy3/600 channels. The amplification number of probes and dye choice for each target and labeling scheme are listed in the two tables below.

| | | | Cy5 or Cy5.5 | | Cy3 or Alexa546 | | |
|---|---|---|---|---|---|---|---|
| | | | # of | # of | # of | # of | Intensity |
| | | | repeats on | repeats on | repeats on | repeats on | Code |
| Labeling | | Dye | primary | secondary | primary | secondary | (Cy3/600: |
| Scheme | Target | choice | amplifier | amplifier | amplifier | amplifier | Cy5/700) |
| 3 | CD9 | Cy3, | 4 | 4 | 4 | 4 | 1:2 |
| | | Cy5 | | | | | |
| | CD34 | Alexa546, | 4 | 4 | 4 | 4 | 2:1 |
| | | Cy5.5 | | | | | |
| | CD45 | Cy5 | 4 | 4 | 0 | 0 | 0:2 |
| | Pancyto | Cy3 | 0 | 0 | 4 | 4 | 1:0 |
| | keratin | | | | | | |

With the probe design and intensity codes used in the tables above, intensity level 1 using the Labeling Scheme-3 was defined as 96 (6*4*4) Cy5.5 or Cy3 dyes in Cy5 or Cy3 channel. Intensity level 2 using the Labeling Scheme-3 was defined as 96 Cy5 or Alexa546 dyes in Cy5 or Cy3 channel.

Synthesis of oligo probes: All the oligo sets were ordered from IDT in a tube or plate format.

Antibody oligo conjugation: The conjugation process consists of three main steps: antibody activation, oligo activation, and antibody-oligo conjugation.

Depending how the conjugation of oligos to antibodies can affect the antigen binding activity of the antibodies, oligos can be conjugated to antibodies through multiple ways such as (but not limited to) conjugation to the amine groups, carboxyl group, sulfhydryl group. The binding capacity of antibody to its specific antigens on the target protein is tested to make sure oligo conjugation doesn't disrupt or has the minimal effect on the antibody-antigen interaction. In this example, antibodies are conjugated to oligos by copper-free click chemistry between dibenzocyclooctyne (DBCO) and azide.

Antibody activation: In this example, oligos are conjugated to antibodies through the amine group on the antibodies. Purified antibodies are adjusted to a concentration of 1 mg/ml with PBS. DBCO-NHS ester dissolved in anhydrous DMSO is added to purified antibody in various molar excesses. Unreacted and excessive DBCO-NHS is removed by gel filtration columns.

Oligo activation (to make azide-modified oligo): Amine-modified oligos are dissolved in PBS (pH 7.4) was mixed with excessive 3-azidopropionic acid sulfo-NHS ester (3AA-NHS) dissolved in anhydrous at 25° C. for 2 hours. Excessive 3AA-NHS is removed by gel-filtration using a spin column.

Antibody-oligo conjugation: Excessive activated azide-oligos are mixed with DBCO-antibody-DBCO solution (PBS, pH 7.2) at 25° C. for one hour in a microcentrifuge tube. The conjugate is purified with Conjugate Clean Up Reagent (from Abcam) and centrifugation in a microcentrifuge tube.

An example of protein staining experiment: Fresh frozen human breast cancer tumor tissue slides can be order ordered from vendors. For staining, sections are fixed in 4% PFA solution for 30 min and washed in PBS for 15 min. At this stage, mild 1-h prebleaching with 1% H₂O₂ in PBS could optionally be applied (for TSA). Samples are blocked for 1 h with PBS containing 2% BSA and 0.1% Triton X-100, with three buffer exchanges. DNA-conjugated primary antibodies are diluted in the blocking solution supplemented with 0.2 μg ml-1 sheared salmon sperm DNA, 0.05% dextran sulfate and optionally 4 mM EDTA, and incubated with the samples at room temperature for 1 h. Excess antibodies are removed by washing at room temperature three times for 15 min with PBS containing 2% BSA and 0.1-0.3% Triton X-100, and twice for 5 min with PBS. Bound antibodies are then post-fixed with 5 mM BS(PEG)5 in PBS for 30 min at room temperature, followed by quenching in 100 mM NH₄Cl in PBS for 5 min, and washed for 15 min with PBS with 0.1% Triton X-100 at room temperature. The incubation with secondary probes, primary amplifiers or secondary amplifiers are performed sequentially at 37° C. in 20-30% formamide, 10% dextran sulfate and 0.1% (vol/vol) Tween-20 in 2×SSC with 0.2 mg ml-1 sheared salmon sperm DNA for 1-2 h. Probes are diluted in this buffer at a final concentration of 100 nM. For multiplexing, all probes in the same tier of signal amplification are incubated simultaneously. After each tier of probe hybridization, the samples are washed for 5 min at room temperature with 50% formamide in PBS and three times for 10 min each with PBS+0.1% Triton X-100 at 37° C.

Imaging setup, and imaging process can be conducted in the same way as described in Example 1 and 2. Cell segmentation can be done manually using ImageJ. The average intensity per pixel in the stained region of each cell is then extracted for intensity ratio analysis. The unstained regions in each cell is excluded for intensity ratio analysis. In this way, each cell can be given an intensity ratio according to their average intensity of the stained regions in 700 and 600 channels. Then the 2D intensity distribution of all cells of interest can be plotted in the same way as that in Example 4 for RNA FISH. The same approach of assigning intensity codes based on the reference intensity code map can be used here as well.

### Example 16

### Multiplex Protein Detection In Vitro with Microarray

This example demonstrates the application of high capacity intensity ratio barcoding for protein detection in vitro. Different proteins are attached onto glass slides at different spatial locations to form a microarray.

Here, we tested using 2 color channels and 2 intensity levels to detect 4 proteins: CD34, CD45, CD9, Pancytokeratin on microarray. We used 4×4 branched DNA amplification and the same set of probes for the Labeling Scheme-3 in Example 15 to amplify the signal and encode these 4 proteins. Different proteins were detected by their antibodies conjugated with protein-specific oligos working as primary probes to bind with intensity coded oligo probes. 4 imaging probes were used to detect 4 proteins: imaging probe-1 for Pancytokeratin was labeled with Cy3 dye only as the intensity code of 1:0, imaging probe-2 for CD45 was labeled with Cy5 dye only as the intensity code of 0:2, imaging probe-3 for CD9 was labeled with Cy5 and Cy3 dye at 5′ and 3′ end respectively as the intensity code of 1:2, imaging probe-4 for CD34 was labeled with Cy5.5 dye and Alexa546 dye at 5′ and 3′ end respectively as the intensity code of 2:1. The first digit of all four intensity codes here represents the intensity level in Cy3/600 channel, the second digit in Cy5/700 channel.

Array preparation and protein staining: Primary antibodies for these 4 proteins were spotted onto a glass slide with surface treatment to attach antibodies well in a spatially separated pattern. Each spot was around 1 mm size and any two spots were separated by 2 cm. Each protein had 10 spots. So in total, 40 spots were formed to capture 4 different proteins. Purified proteins were dissolved in a PBS buffer in 1 mg/ml and incubated with the spotted slide for at least 1 hour at room temperature. Then primary antibodies conjugated with coded oligos were incubated with the slide for another 1 hour and excessive antibodies were washed away with washing buffer at room temperature. After that, multiple tiers of intensity coded probes (100 nM) were incubated with the slide with captured proteins. Each tier of probe labeling lasted for 1 hour at room temperature and excessive probes were washed away with the washing buffer. Finally, the slide was scanned by PMT with 2 color channels (Cy5/700 nm channel, Cy3/600 nm channel). The 700 channel was excited by 640 nm laser and installed with an emission filter of 700/75 m. The 600 channel was excited by 561 nm laser and installed with an emission filter of 600/50 m.

Image analysis: The average intensity of individual protein spots was analyzed and plotted in a 2D intensity distribution map. Then protein spots with similar intensity ratios were clustered together. Similar to the Example-4 for RNA FISH, each cluster was fitted with a reference intensity code map and then assigned with the best intensity code. In this way, proteins were successfully detected.

### ASPECTS OF THE DISCLOSURE

Aspect 1. A sample prepared for examination, comprising: a first plurality of probes bound, directly or indirectly, to a first target molecule in a biological sample, and a second plurality of probes bound, directly or indirectly, to a second target molecule in the biological sample, wherein each of the probes is attached to one or more kind of optical labels such that: (a) a first kind of optical label is associated with the first target molecule, and (b) a second kind of optical label is associated with the second target molecule, wherein each target is associated with at least two kinds of optical labels, the first plurality of probes is attached with at least the first kind of optical labels, the second plurality of probes is attached with at least the second kind of optical labels, and wherein the first and second target molecules, upon excitation, are associated with different ratios of signal intensities from two or more than two color channels.

Aspect 2. The sample of aspect 1, wherein the first kind of optical label is associated with both the first and second target molecule, the second kind of optical label is associated with both the first and second target molecule, the ratio of the number of the first kind of optical label to the number of the second kind of optical label associated with the first target molecule and this ratio associated with the second target molecule differ by at least a factor of 2, 2.3, 3.5, 5, 8.1, 10, 20, 50, or 100.

Aspect 3. The sample of aspect 2, wherein each probe in the first and second plurality of probes is only associated with one kind of optical label, and the number of the first plurality of probes for the first target is different from the number of the second plurality of probes for the second target.

Aspect 4. The sample of aspect 1 and 2, wherein the first and second kind of optical labels are respectively having the same or partially overlapping color spectra but different intensity in a first color channel, the first target is associated with a third kind of optical label.

Aspect 5. The sample of aspect 4, wherein the second target is associated with a fourth kind of optical label, the third and fourth kind of optical labels are respectively having the same or partially overlapping color spectra but different intensity in a second color channel.

Aspect 6. The sample of aspects 4 and 5, wherein the first and second kinds of optical labels are in different number.

Aspect 7. The sample of aspect 6, wherein the first and second plurality of probes are in different number.

Aspect 8. The sample of any preceding aspect, wherein a third plurality of probes bind, directly or indirectly, to both the first and second target molecules in the biological sample, and each of the third plurality of probes is attached with a fifth kind of optical label.

Aspect 9. The sample of any preceding aspect, wherein different kinds of optical labels are attached with different probes in a plurality of probes associated with the same target molecule.

Aspect 10. The sample of aspect 9, wherein different probes associated with different optical labels bind to a target molecule in an alternating order.

Aspect 11. The sample of any preceding aspect, wherein any plurality of probes associated with a target molecule are comprised of at least 2 probes.

Aspect 12. The sample of aspect 11, wherein the first or second plurality of probes associated with a target molecule have at least 12 probes.

Aspect 13. The sample of any preceding aspect, wherein the first or second target molecule is associated with at least 3 different kinds of optical labels and detected by at least 3 color channels.

Aspect 14. The sample of any preceding aspect, where each probe in a plurality of probes is attached to two or more different kinds of optical labels or two or more different numbers of the same optical labels.

Aspect 15. The sample of any preceding aspect, wherein at least one optical label is attached with a quenching or signal enhancing molecule.

Aspect 16. The sample of any preceding aspect, wherein at least one optical label is attached with more of the same optical label, or with a fifth kind of optical label.

Aspect 17. The sample of any preceding aspect, wherein at least two of the same optical labels associated with the same target are separated by no more than 10 nucleotides along the probe sequence.

Aspect 18. The sample of aspects 1-5, wherein the first or second plurality of probes attached with optical labels are associated with a target molecule through primary probes.

Aspect 19. The sample of aspects 18, wherein the first or second plurality of the probes are further associated with at least one tier of intermediate probes after the primary probes are associated with a target molecule.

Aspect 20. The sample of aspect 19, wherein the first or second plurality of the probes are further associated with primary amplifiers and secondary amplifiers after the primary probes are associated with a target molecule.

Aspect 21. The sample of aspects 18-20, wherein the first target is associated with both the first and second kinds of optical labels, and each primary probe on a target are associated with both the first and second kinds of optical labels.

Aspect 22. The sample of aspect 21, wherein the first and second kinds of optical labels associated with the same primary probe are in equal number.

Aspect 23. The sample of aspect 22, wherein the first and second kinds of optical labels associated with the same primary probe are in different number.

Aspect 24. The sample of aspect 22 and 23, wherein the first and second kinds of optical labels associated the same primary probe are labeled at different ends of the primary probe, either 5′ or 3′ end.

Aspect 25. The sample of aspects 18-20, wherein the first target molecule is associated with both the first and second kinds of optical labels, and they are in different number.

Aspect 26. The sample of aspect 25, wherein different kinds of optical labels detecting the same target molecule are associated with different primary probes.

Aspect 27. The sample of aspects 18-27, wherein the first kind of optical label is associated with both the first and second target molecule, and the ratio of the number of the first optical label for the first target molecule and the number of the first optical label for the second target molecule is at least 2, 2.5, 3, 3.3, 4, 4.2, 5, 8, 8.9 or 10.

Aspect 28. The sample of aspects 18 and 19, wherein the first or second plurality of the probes are associated with their primary probes by enzymatic signal amplification, such as rolling cycle amplification.

Aspect 29. The sample of any preceding aspect, wherein the probes are single-stranded oligonucleotides or peptides.

Aspect 30. The sample of any preceding aspect, wherein the optical labels are fluorescent dyes, fluorescent proteins or nanoparticles.

Aspect 31. The sample of any preceding aspect, wherein the target molecules comprise RNA molecules, RNA fragments, DNA molecules of no more than 100 kb, or DNA fragments of no more than 100 kb.

Aspect 32. The sample of any one of aspects 1-31, wherein the target molecules comprise proteins, lipids, polysaccharides, or particles.

Aspect 33. The sample of any one of aspects 1-31, wherein the target molecules comprise DNA modifications, RNA modifications or protein modifications.

Aspect 34. The sample of any one of aspects 32 and 33, wherein the probes attached with optical labels are associated with the target molecules through oligonucleotides or peptides.

Aspect 35. The sample of any preceding aspect, wherein the target molecules are located in a cell, a tissue, or attached on a solid scaffold.

Aspect 36. A kit, package, or mixture of probes for hybridization, comprising: a first plurality of probes each of which can bind to a first target molecule, or a first plurality of probes and one or more intermediate probes which allow the first plurality of probes to bind indirectly to the first target molecule, and a second plurality of probes each of which can bind to a second target molecule, or a second plurality of probes and one or more intermediate probes which allow the second plurality of probes to bind indirectly to the second target molecule, wherein the first plurality of probes is attached with at least a first kind of optical labels, the second plurality of probes is attached with at least a second kind of optical labels, each target is associated with at least two kinds of optical labels, wherein the first and second target molecules, upon excitation, are associated with different ratios of signal intensities from two or more than two color channels.

Aspect 37. A method of detecting two or more target molecules in a sample, comprising admixing the probes of aspect 36 to a sample that comprises the first and second target molecules under conditions to allow the probes to bind to the target molecules, wherein different kinds of target molecules are associated with different combination of optical labels, each target is detected by at least 2 color channels to create an intensity ratio, different targets are differentiated by different intensity ratios.

Aspect 38. The method of any one of aspects 37, wherein the different intensity ratios associated with the target molecules are matched with a reference intensity code map to allow the detection of the different kinds of target molecules.

Aspect 39. The method of any one of aspects 38, wherein a portion of intensity ratio codes that are available from a reference intensity code map are used to design the probes.

Aspect 40. The method of aspect 38 and 39, wherein the detection comprises comparing detected intensity ratios to the distribution of intensity codes in a reference intensity code map.

Aspect 41. The method of aspect 40, wherein the comparison comprising assigning the detected intensity ratio to an intensity code in the reference intensity code map.

Aspect 42. The method of aspect 41, wherein a detected intensity ratio is rejected if the directed intensity ratio is farther than a predetermined cutoff value from any intensity code in the intensity code map.

Aspect 43. The method of any one of aspects 37-42, wherein any two target molecules are spatially separated by at least 250 nm.

Aspect 44. A sample prepared for examination, comprising: a first plurality of probes attached with a first kind of optical label, bound, directly or indirectly, to a first target molecule in a biological sample, and a second plurality of probes attached with a second kind of optical label, bound, directly or indirectly, to a second target molecule in the biological sample, wherein the first plurality of probes is attached with the first kind of optical labels, the second plurality of probes is attached with the second kind of optical labels, different targets are associated with different kinds of optical label, the first and second kind of optical labels are having overlapped excitation or emission spectra in a first and second color channels, wherein the first and second target molecules, upon excitation, are associated with different ratios of signal intensities from two color channels.

Aspect 45. The sample of aspect 44, wherein each probe is a single-strand oligonucleotide, peptide or a hybrid of oligo and peptide.

Aspect 46. The sample of aspects 44 and 45, wherein the number of the first optical label associated with the first target molecule is different from the number of the first optical label associated with the second target molecule.

Aspect 47. The sample of aspects 45 and 46, wherein the first optical label for the first target and the second optical label for the second target are associated with different number of primary probes.

Aspect 48. The sample of aspects 45-47, wherein each plurality of probes are comprised of at least 2 probes.

Aspect 49. The sample of aspects 48, wherein each plurality of probes are comprised of at least 12 probes.

Aspect 50. The sample of any one of aspects 44-49, wherein any two target molecules are spatially separated by at least 250 nm.

Aspect 51. The sample of any one of aspects 44-50, wherein a third plurality of probes attached with a second kind of optical label, bound, directly or indirectly, to a third target molecule in the biological sample, a third optical label is associated with the third target, the third optical label has overlapped spectrum with the first and second optical labels in both the first and second color channels.

Aspect 52. The sample of any one of aspects 51, wherein the third target molecules, upon excitation, are associated with different ratio of signal intensities from the two color channels.

Aspect 53. A kit, package, or mixture of probes for hybridization, comprising: a first plurality of probes each of which can bind to a first target molecule, or a first plurality of probes and one or more intermediate probes which allow the first plurality of probes to bind indirectly to the first target molecule, and a second plurality of probes each of which can bind to a second target molecule, or a second plurality of probes and one or more intermediate probes which allow the second plurality of probes to bind indirectly to the second target molecule, wherein the first plurality of probes is attached with a first kind of optical labels, the second plurality of probes is attached with a second kind of optical labels, different targets are associated with different kinds of optical label, the first and second kind of optical labels are having overlapped excitation or emission spectra in a first and second color channel, wherein the first and second target molecules, upon excitation, are associated with different ratios of signal intensities from two color channels.

Aspect 54. A method of detecting two or more target molecules in a sample, comprising admixing the probes of aspect 53 to a sample that comprises the first and second target molecules under conditions to allow the probes to bind to the target molecules, wherein different kinds of target molecules are associated with different optical labels, each target is detected by at least 2 color channels to create an intensity ratio, different targets are differentiated by different intensity ratios.

Aspect 55. The method of any one of aspects 54, wherein the different intensity ratios associated with the target molecules are matched with a reference intensity code map to allow the detection of the different kinds of target molecules.

Aspect 56. The method of any one of aspects 55, wherein a portion of intensity ratio codes that are available from a reference intensity code map are used to design the probes.

Aspect 57. The method of any one of aspects 54-56, wherein the detection comprises comparing detected intensity ratios to the distribution of intensity codes in a reference intensity code map.

Aspect 58. The method of aspect 57, wherein the comparison comprising assigning the detected intensity ratio to an intensity code in the reference intensity code map.

Aspect 59. The method of aspect 58, wherein a detected intensity ratio is rejected if the directed intensity ratio is farther than a predetermined cutoff value from any intensity code in the intensity code map.

Aspect 60. The method of any one of aspects 54-59, wherein any two target molecules are spatially separated by at least 250 nm.

Aspect 61. A sample prepared for examination, comprising: a first plurality of imaging probes attached with a first kind of optical label, bound, directly or indirectly, to a first target molecule in a biological sample, and a second plurality of imaging probes attached with the first kind of optical label, bound, directly or indirectly, to a second target molecule in the biological sample, wherein the ratio of the number of optical labels associated with the first plurality of imaging probes and the number of optical labels associated with the second plurality of imaging probes is more than 2, wherein the first and second target molecules, upon excitation, are associated with different ratios of signal intensities from a first color channel.

Aspect 62. The sample of aspect 61, wherein the first plurality of imaging probes are further associated with a first plurality of primary probes, the first plurality of primary probes are associated with the first target molecule, and each probe of the primary probes is associated with more than one optical labels directly or indirectly.

Aspect 63. The sample of aspects 61 and 62, wherein the first and second target molecules are separated by at least 20 nm, 100 nm or 250 nm.

Aspect 64. The sample of aspect 61, wherein the ratio of the number of optical labels associated with the first plurality of imaging probes and the number of optical labels associated with the second plurality of imaging probes is at least 3, 4, or 5.

Aspect 65. A kit, package, or mixture of probes for hybridization, comprising: a first plurality of probes each of which can bind to a first target molecule, or a first plurality of probes and one or more intermediate probes which allow the first plurality of probes to bind indirectly to the first target molecule, and a second plurality of probes each of which can bind to a second target molecule, or a second plurality of probes and one or more intermediate probes which allow the second plurality of probes to bind indirectly to the second target molecule, wherein the first and second plurality of probes are both attached with a first kind of optical label but in different number, wherein the first and second target molecules, upon excitation, are associated with different signal intensities from the same color channel.

Aspect 66. A method of detecting two or more target molecules in a sample, comprising admixing the probes of aspect 65 to a sample that comprises the first and second target molecules under conditions to allow the probes to bind to the target molecules, wherein different kinds of target molecules are associated with the same kind of optical label but different number, any two target molecules are spatially separated by at least 20 nm, the different intensities associated with the target molecules are matched with a reference intensity code map to allow the detection of the different kinds of target molecules.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A biological sample prepared for examination, comprising:
a first target molecule bound, directly or indirectly, to a first optical label, and
a second target molecule bound, directly or indirectly, to a second optical label,
wherein the first target molecule and the second target molecule are optically distinguishable in one or more color channels by virtue of (a) different numbers of optical labels bound to each if the first optical label is the same as the second optical label, or (b) different intensities of similar color between the first optical label and the second optical label.

2. The biological sample of claim 1, wherein the first optical label is the same as the second label, and wherein the first target molecule is further bound to a third optical label different from the first and second optical label.

3. The biological sample of claim 2, wherein the first target molecule is bound to a first probe comprising a first number of the first optical label, and the third optical label.

4. The biological sample of claim 3, wherein the second target molecule is bound to a second probe comprising a second number of the second optical label, the second number being different from the first number.

5. The biological sample of claim 4, wherein the second probe further comprises the third optical label.

6. The biological sample of claim 1, wherein the first optical label and the second optical label have peak emission wavelengths separated by <100 nm, <50 nm, or <20 nm.

7. A biological sample prepared for examination, comprising a plurality of distinct target molecules, each of which is bound to one or more optical labels, wherein the target molecules is optically distinguishable from one another in one or more color channels, and at least two of the target molecules are bound to the same optical labels but are optically distinguishable due to different ratios of the different optical labels bound to the target molecule.

8. The sample of claim 7, wherein the ratios differ by at least a factor of 2, 2.3, 3.5, 5, 8.1, 10, 20, 50, or 100.

9. The sample of claim 7, wherein the at least two target molecules each is bound to at least three different optical labels.

10. The sample of claim 7, wherein each of the target molecules is bound to one or more probes, and the probes bound to one target molecule is optically distinguishable from probes bound to each of the other target molecules.

11. The sample of claim 7, wherein each of the target molecules is bound to one or more probes, and the probes bound to one target molecule is not necessarily optically distinguishable from probes bound to each of the other target molecules, but the combination of all probes bound to a target molecule enables the target molecule to be optically distinguishable from other target molecules.

12. A biological sample prepared for examination, comprising a plurality of distinct target molecules, each of which is bound to one or more optical labels, wherein the target molecules is optically distinguishable from one another in one or more color channels, and at least two of the target molecules are bound to one common first optical label and, respectively, a second optical label and a third optical label having similar color, but are optically distinguishable due to the second and third optical labels having different intensities.

13. The sample of claim 12, wherein the at least two target molecules are bound to different numbers of the first optical label.

14. The sample of claim 12, wherein each of the target molecules is bound to one or more probes, and the probes bound to one target molecule is optically distinguishable from probes bound to each of the other target molecules.

15. The sample of claim 12, wherein the second optical label and the third optical label have peak emission wavelengths separated by <100 nm, <50 nm, or <20 nm.

16. The sample of claim 1, wherein at least one target molecule is further bound to a quenching molecule or a signal enhancing molecule.

17. The sample of claim 1, wherein at least two of the same optical labels associated with the same target molecule are in proximity enough to cause signal quenching.

18-25. (canceled)

26. A kit, package, or mixture of probes for hybridization, comprising probes labeled with optical labels suitable for preparing a sample of claim 1.

27. A method of detecting two or more target molecules in a sample, comprising admixing the probes of claim 26 to a sample that comprises target molecules under conditions to allow the probes to bind to the target molecules, wherein different kinds of target molecules are associated with different combinations of optical labels, each target molecule is detected by at least two color channels.

28. The method of claims 27, wherein different optical signals associated with the optical labels associated with the target molecules are matched with a reference optical code map to allow detection of the different target molecules.

29-37. (canceled)


SEQUENCE LISTING (13 sequences)

SEQ ID NO: 1 - DNA, 18 bp, Artificial Sequence
  Synthetic
ccctaaccctaaccctaa

SEQ ID NO: 2 - DNA, 17 bp, Artificial Sequence
  Synthetic
attcgttggaaacggga

SEQ ID NO: 3 - DNA, 17 bp, Artificial Sequence
  Synthetic
attcgttggaaacggga

SEQ ID NO: 4 - DNA, 17 bp, Artificial Sequence
  Synthetic
attcgttggaaacggga

SEQ ID NO: 5 - DNA, 17 bp, Artificial Sequence
  Synthetic
attcgttggaaacggga

SEQ ID NO: 6 - DNA, 20 bp, Artificial Sequence
  Synthetic
  misc_feature (1)..(20): n is a, c, g, or t
nnnnnnnnnnnnnnnnnnnn

SEQ ID NO: 7 - DNA, 20 bp, Artificial Sequence
  Synthetic
  misc_feature (1)..(20): n is a, c, g, or t
nnnnnnnnnnnnnnnnnnnn

SEQ ID NO: 8 - DNA, 20 bp, Artificial Sequence
  Synthetic
  misc_feature (1)..(20): n is a, c, g, or t
nnnnnnnnnnnnnnnnnnnn

SEQ ID NO: 9 - DNA, 20 bp, Artificial Sequence
  Synthetic
  misc_feature (1)..(20): n is a, c, g, or t
nnnnnnnnnnnnnnnnnnnn

SEQ ID NO: 10 - DNA, 20 bp, Artificial Sequence
  Synthetic
  misc_feature (1)..(20): n is a, c, g, or t
nnnnnnnnnnnnnnnnnnnn

SEQ ID NO: 11 - DNA, 20 bp, Artificial Sequence
  Synthetic
  misc_feature (1)..(20): n is a, c, g, or t
nnnnnnnnnnnnnnnnnnnn

SEQ ID NO: 12 - DNA, 20 bp, Artificial Sequence
  Synthetic
  misc_feature (1)..(20): n is a, c, g, or t
nnnnnnnnnnnnnnnnnnnn

SEQ ID NO: 13 - DNA, 21 bp, Artificial Sequence
  Synthetic
ccaggtgagcatctgacagcc